(19) United States
(12) Patent Application Publication
(10) Pub. No.: US 2008/0057050 A1
(43) Pub. Date: Mar. 6, 2008

(54) Intravenous injection of plasminogen non-neurotoxic activators for treating cerebral stroke

(75) Inventors: Mariola Sohngen, Aachen (DE); Wolfgang Sohngen, Aachen (DE); Dirk Eyding, Valby (DK); Yasir Alaa Shafeek Al-Rawi, Aachen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Paion Deutschland GmbH

(21) Appl. No.: 11/878,686

(22) Filed: Jul. 26, 2007

Related U.S. Application Data
(63) Continuation of application No. 11/264,088, filed on Nov. 2, 2005, now abandoned.
(63) Continuation-in-part of application No. PCT/EP2004/004626, filed on Apr. 30, 2004.
Application No. PCT/EP2004/004626 is a continuation-in-part of application No. PCT/EP2003/004729, filed on May 6, 2003.
Application No. PCT/EP2004/004626 is a continuation-in-part of application No. PCT/EP2003/004608, filed on May 2, 2003.
(60) Provisional application No. 60/627,641, filed on Nov. 12, 2004.

(51) Int. Cl. A61K 38/48; A61P 43/00

(52) U.S. Cl. 424/94.63

(57) ABSTRACT

The invention refers to the use of non-neurotoxic plasminogen activating factors, e.g., from Desmodus rotundus (DSPA) or genetically modified plasminogen activating factors, for example of human origin, for the therapeutic treatment of stroke in mammals.

This application claims the benefit of the filing date of U.S. Provisional Application No. 60/627,641, filed on Nov. 12, 2004. This application is also a continuation in part of PCT Application No. PCT/EP2004/004626, filed on Apr. 30, 2004, which is a continuation in part of PCT Application No. PCT/EP2003/004729, filed on May 6, 2003; and of PCT Application No. PCT/EP2003/004608 filed on May 2, 2003.

The invention refers to the intravenous application of non-neurotoxic plasminogen activators, including genetically modified plasminogen activators and plasminogen activators from the saliva of Desmodus rotundus (DSPA) for the treatment of stroke in mammals, such as humans. The treatment of stroke with these plasminogen activators is known from international patent application PCT/EP02/12204 (US 2005/0048027).

Clinical Characteristics and Biochemistry of Stroke

Different clinical pictures are summarized under the term “stroke,” which correlate in their clinical symptoms. According to the respective pathogenesis, a first differentiation between these clinical pictures in so called ischaemic and haemorrhagic insults is possible.

Ischaemic insults (ischaemia) are characterized in a reduction or interruption of the blood circulation in the brain due to a lack of arterial blood supply. Often this is caused by thrombosis of an arteriosclerotic stenosed vessel or by arterio arterial cardial embolisms respectively.

Haemorrhagic insults are based, inter alia, on the perforation of brain supplying arteries damaged by arterial hypertonia. However, only approximately 20% of all cerebral insults are caused by haemorrhagic insults. Thus, stroke due to thrombosis is much more relevant.

In comparison to other tissue ischaemias, the ischaemia of the neuronal tissue is widely accompanied by necrosis of the affected cells. The higher incidence of necrosis in neuronal tissue can be explained with the new understanding of the phenomenon “excitotoxicity,” which is a complex cascade comprising a plurality of reaction steps. The cascade is initiated by ischaemic neurons affected by a lack of oxygen, which then lose ATP instantaneously and depolarize. This results in an increased postsynaptic release of the neurotransmitter glutamate, which activates membrane bound glutamate receptors regulating cation channels. However, due to the increased glutamate release, glutamate receptors become over activated.

Glutamate receptors regulate voltage dependent cation channels which are opened by a binding of glutamate to the receptor. This results in a Na⁺ and Ca²⁺ influx into the cell massively disturbing the Ca²⁺ dependent cellular metabolism. The activation of the Ca²⁺ dependent catabolic enzymes could give reason to the subsequent cell death (Lee, Jin-Mo et al., “The changing landscape of ischaemic brain injury mechanisms” Nature, Supp. to Vol. 399, No. 6738, (Jun. 24, 1999), pp. A7-A14; Dennis W. Choi “Glutamate neurotoxicity and diseases of the nervous system” Neuron, Vol. 1, (October, 1998), pp. 623-634).

Although the mechanism of glutamate mediated neurotoxicity is not yet entirely understood, it is agreed upon that it contributes in a large extent to the neuronal cell death following cerebral ischaemia (Jin-Mo Lee, et al.).

Therapy of Stroke

Besides safeguarding vital functions and stabilizing physiological parameters, the re-opening of the closed vessel has priority in the therapy of acute cerebral ischaemia. The re-opening can be performed by different means. The mere mechanical re-opening, as e.g. the PTCA after heart attack, so far has not yet led to satisfying results. Only with a successful fibrinolysis, can an acceptable improvement of the physical condition of patients be achieved. This can be accomplished by a local application using a catheter (PROCAT, a study with pro-urokinase). However, despite first positive results, this method has not yet been officially approved as a pharmaceutical treatment.

The naturally occurring fibrinolysis is based on the proteolytic activity of the serine protease plasmin, which originates from its inactive precursor by catalysis (activation). The natural activation of plasminogen is catalyzed by the plasminogen activators u-PA (urokinase type plasminogen activator) and t-PA (tissue plasminogen activator) occurring naturally in the body. In contrast to u-PA, t-PA forms a so called activator complex together with fibrin and plasminogen. Thus, the catalytic activity of t-PA is fibrin dependent and is enhanced in its presence approximately 550-fold. Besides fibrin, fibrinogen can also stimulate t-PA mediated catalysis of plasminogen to plasmin—even though to a smaller extent. In the presence of fibrinogen the t-PA activity is only increased 25-fold. Also, the cleavage products of fibrin (fibrin degradation products (FDP)) are stimulating t-PA.

Known Therapeutic Approaches

a) Streptokinase

Early attempts of thrombolytic treatment of acute stroke go back to the 1950s. First extensive clinical trials with streptokinase, a fibrinolytic agent from beta-haemolysing streptococci, started only in 1995. Together with plasminogen, streptokinase forms a complex that catalyzes other plasminogen molecules into plasmin.

The therapy with streptokinase has severe disadvantages since it is a bacterial protease and therefore can provoke allergic reactions in the body. Furthermore, due to a former streptococci infection including a production of antibodies, the patient may exhibit a so called streptokinase resistance making the therapy more difficult. Besides this, clinical trials in Europe (Multicenter Acute Stroke Trial of Europe (MAST-E), Multicenter Acute Stroke Trial of Italy (MAST-I)) and Australia (Australian Streptokinase Trial (AST)) indicated an increased mortality risk and a higher risk of intracerebral bleeding (intracerebral hemorrhage, ICH) after treating patients with streptokinase. These trials had to be terminated early.

b) Urokinase

Alternatively, urokinase—also a classical fibrinolytic agent—can be used. In contrast to streptokinase, it does not exhibit antigenic characteristics since it is an enzyme naturally occurring in various body tissues. It is an activator of plasminogen and independent of a co-factor. Urokinase is produced in kidney cell cultures.

c) recombinant t-PA (rt-PA)

Extensive experience on therapeutic thrombolysis is available for recombinant tissue type plasminogen activator—the so called rt-PA—(see EP 0 093 619, U.S. Pat. No. 4,766,075), which is produced in recombinant hamster cells. In the 90s, several clinical trials were performed world-wide using t-PA—with acute myocardial infarction as the main indication—leading to partially non-understood and contradictory results. In the so called European Acute Stroke Trial (ECASS), patients were treated within a time frame of 6 hours after the onset of the symptoms of a stroke intravenously with rt-PA. After 90 days the mortality rate as well as the Barthel-Index were examined as an Index for the disability or the independent viability of patients. No significant improvement of the viability was reported but an—even though not significant—increase of mortality was observed. Thus, it could be concluded, a thrombolytic treatment with rt-PA of patients being individually selected according to their respective case history immediately after the beginning of the stroke could possibly be advantageous. However, a general use of rt-PA within the time frame of 6 hours after the onset of stroke was not recommended since an application during this time increases the risk of intracerebal hemorrhage (C. Lewandowski C and Wiliam Barsan, 2001: Treatment of Acute Stroke; in: Annals of Emergency Medicine 37:2; S. 202 ff.).

The thrombolytic treatment of stroke was also subject of a clinical trial conducted by the National Institute of Neurologic Disorder and Stroke (so called NINDS rtPA Stroke Trial) in the United States. This trial concentrated on the effect of intravenous rt-PA treatment within only three hours after the onset of the symptoms. Patients were examined three months after the treatment. Due to the observed positive effects of this treatment on the viability of patients, rt-PA treatment within these limited time frame of three hours was recommended, although the authors found a higher risk for ICH.

Two further studies (ECASS II Trial: Alteplase Thrombolysis for Acute Noninterventional Therapy in Ischaemic Stroke (ATLANTIS)) examined whether the positive effects of rt-PA treatment within three hours after the onset of stroke could be repeated even with a treatment within six hours time. However, this question could not be answered affirmatively since no improvement of the clinical symptoms or any decrease in mortality was observed. The higher risk for ICH remained.

According to a review of all stroke trials first published in 1997 and updated in March 2001, all treatments with thrombolytics (urokinase, streptokinase, rt-PA or recombinant urokinase) resulted in a significant higher mortality within the first 10 days after the stroke while the total number of either dead or disabled patients was reduced when the thrombolytics where applied within six hours after stroke onset. These effects were mainly due to ICH. The broad use of thrombolytics for the treatment of stroke was therefore not recommended.

Even before, such results gave reason to some other authors mere sarcastic statement that stroke patients had the choice to either die or to survive disabled (“Thrombolysis in stroke not justified”, SCRIP 1997: 2265, 26).

Nevertheless, so far the therapy with rt-PA is the only treatment of acute cerebral ischaemia approved by the Food and Drug Administration (FDA) in the United States. However, it is restricted to an application of rt-PA within three hours after the onset of stroke.

Recombinant plasminogen activator is currently marketed under the designation Alteplase or Reteplase for the analogous drug. The latter is a therapeutically active t-PA fragment with a lower half-life period. The therapeutic dose for Alteplase is about 70-100 mg, for Reteplase 2×560 mg, whereas Alteplase is mainly applied via a drip infusion and Reteplase via a bolus injection repeated twice in an interval of about 30 min (Mutschler: “Arzneimittelwirkungen”, 8^{th} Edition, pages 512-513).

Side Effects of t-PA

Section 1.01 Neurotoxicity and Excitotoxicity

The approval of rt-PA was reached in 1996. Before, in the year 1995, first announcements about negative side effects of t-PA became known, which highlight the surprising therapeutic effect obtained when administering the plasminogen activators of the invention for stroke treatment outside the three-hour time frame. It was known, for example, that microglia cells and neuronal cells of the hippocampus produce t-PA, which contributes to the glutamate mediated excitotoxicity. This is concluded from a comparative study on t-PA deficient and wild type mice when glutamate agonists were injected in their hippocampus, respectively. The t-PA deficient mice showed a significant higher resistance against external (inthrathecal) administered glutamate (Tsirka S E et al., Nature, Vol. 377, 1995, “Excitoxin-induced neuronal degeneration and seizure are mediated by tissue plasminogen activator”). These results were confirmed in 1998 when Wang et al. could prove nearly a double quantity of necrotic neuronal tissue in t-PA deficient mice when t-PA was injected intravenously. This negative effect of external t-PA on wild type mice was only approximately 33% (Wang et al., 1998, Nature, “Tissue plasminogen activator (t-PA) increases neuronal damage after focal cerebral ischaemia in wild type and t-PA deficient mice”.)

Further results on the stimulation of excitotoxicity by t-PA were published by Nicole et al. in the beginning of 2001 (Nicole O., Docagne F Ali C; Margaill I; Carmeliet P; MacKenzie E T, Vivien D and Buisson A, 2001: The proteolytic activity of tissue-plasminogen activator enhances NMDA receptor-mediated signaling; in Nat Med 7, 59-64). They could prove that t-PA being released by depolarized cortical neurons could interact with the so called NR1 sub-unit of the glutamate receptor of the NMDA type leading to a cleavage of NR1. This increases the receptor's activity resulting in a higher tissue damage after glutamate agonist NMDA was applied. The NMDA agonist induced excitotoxicity. Thus, t-PA exhibits a neurotoxic effect by activating the glutamate receptor of the NMDA type. Since the blood-brain barrier breaks down in the affected tissue area during a stroke, soluble plasma proteins like fibrinogen as well as therapeutically applied t-PA get into contact with the neural tissue, where the t-PA, being stimulated by the fibrinogen, displays its excitotoxic effect via the activation of the glutamate receptor.

Despite its neurotoxic side effect and its increasing effect on the mortality, t-PA was approved by FDA. This can only be explained by the lack of harmless and effective alternatives—thus, it is due to a very pragmatic cost benefit analysis. Therefore, there is still a need for safe therapies. However, if they were still based on thrombolytics—in case it is not possible to find alternatives to thrombolysis—the problem of neurotoxicity still has to be considered (see for example Wang et al.; Lewandowski and Barson 2001).

Therefore, further examination of known thrombolytics, including DSPA (Desmodus rotundus Plasminogen Activator), in order to develop new drugs for stroke was terminated. In the case of DSPA, its potential suitability for this medical indication was pointed out earlier (Medan P; Tatlisumak T; Takano K; Carano R A D; Hadley S J; Fisher M: Thrombolysis with recombinant Desmodus saliva plasminogen activator (rDSPA) in a rat embolic stroke model; in: Cerebrovasc Dis 1996:6; 175-194 (4^{th} International Symposium on Thrombolic Therapy in Acute Ischaemic Stroke). However, because DSPA is a plasminogen activator with a high homology (resemblance) to t-PA and because of the disillusionment resulting from the neurotoxic side effects of t-PA, there were no further expectations for DSPA to be a suitable drug for stroke treatment.

Alternative Therapeutic Approaches

The investigation of alternative therapeutic approaches is currently focused on, e.g., anticoagulants like heparin, aspirin or ancrod, the active substance derived from the poison of the Malayan pit viper. Two further clinical trials examining the effects of heparin (International Stroke Trial(IST) and Trial of ORG 10172 in Acute Stroke Treatment (TOAST)) however, do not indicate a significant improvement of mortality or a prevention of stroke.

A further new treatment focuses neither on thrombus nor on blood thinning or anti coagulation but attempts to increase the vitality of cells damaged by the interruption of blood supply (WO 01/51613 A1 and WO 01/51614 A1). To achieve this, antibiotics from the group of quinons, aminoglycosides or chloramphenicol are applied. For a similar reason it is further suggested to begin with the application of citicholin directly after the onset of stroke. In the body, citicholin is cleaved to cytidine and choline. The cleavage products form part of the neuronal cell membrane and thus support the regeneration of damaged tissue (U.S. Pat. No. 5,827,832).

Recent research on safe treatment is based on the new finding that a part of the fatal consequences of stroke is caused only indirectly by interrupted blood supply but directly by the excito- or neurotoxicity, including over activated glutamate receptors. This effect is increased by t-PA (see above). A concept to reduce excitotoxicity is therefore to apply so called neuroprotectives. They can be used separately or in combination with fibrinolytic agents in order to minimize neurotoxic effects. They can lead to a reduced excitotoxicity either directly, e.g., as a glutamate receptor antagonist, or indirectly by inhibiting voltage dependent sodium or calcium channels (Jin-Mo Lee et al.).

A competitive inhibition (antagonistic action) of the glutamate receptor of NMDA type is possible, e.g., with 2-amino-5-phosphonovalerate (APV) or 2-amino-5-phosphonoheptanoate (APH). A non competitive inhibition can be achieved, e.g., by substances binding to the phencyclidine side of the channels. Such substances can be phencyclidine, MK-801, dextrorphane or cetamine.

So far, treatments with neuroprotectives have not shown the expected success, possibly because neuroprotectives had to be combined with thrombolytic agents in order to exhibit their protective effects. This applies to other substances.

Even a combination of t-PA and neuroprotective agents results only in a limited damage. Nevertheless, the disadvantageous neurotoxicity of the fibrinolytic agent as such is not avoided.

### BRIEF DESCRIPTION OF THE FIGURES

FIG. 1 shows results indicating that infusion of t-PA or DSPA disperses into the hippocampus of t-PA −/− mice and retains proteolytic activity. See paragraphs [00173] et seq.

FIG. 2 shows results indicating that t-PA and DSPA activity is recovered in hippocampal extracts prepared from t-PA −/− mice following infusion. See paragraphs [00175] et seq.

FIG. 3a shows results indicating that DSPA infusion does not restore kainic-acid mediated neurodegeneration in vivo. See paragraphs [00178] et seq.

FIG. 3b shows serial sections of hippocampal regions for t-PA −/− mice infused with t-PA. See paragraphs [00178] et seq.

FIG. 4 shows brain sections for wild type mice injected with NMDA in the presence of t-PA or DSPA. Results show that injection of NMDA alone caused a reproducible lesion in all treated mice. See paragraphs [00187] et seq.

FIG. 5 shows results indicating that constant relative increase of t-PA neurotoxicity is time limiting for therapy and addiditive to rt-PA neurotoxicity.

FIG. 6 shows results indicating the effect of perfusion/diffusion mismatch on treatment success. See paragraph [00206].

FIG. 7 shows results from various administrations of DSPA and t-PA using the kainic acid model. See paragraphs [0095] et seq.

FIG. 8 shows hippocampal length CA1-3 following kainic acid administration along with various DSPA/t-PA combinations. See paragraphs [0095] et seq.

FIG. 9 shows the amino acid sequence of a modified uroquinase polypeptide (SEQ ID NO: 2). See paragraph [0086].

FIG. 10 shows the amino acid sequence of a modified tissue plasminogen activator (SEQ ID NO: 1). See paragraph [0085].

FIG. 11 (Table 1) shows hippocampal length following kainic acid administration in a tabular form. See paragraphs [0095] et seq.

FIG. 12 (Table 2) shows results from injection of NMDA in conjunction with t-PA and DSPAα1 on wild-type mice. See paragraph [0098].

FIG. 13 (Table 3) shows results from stroke treatment in humans with DSPAα1. See paragraph [0099].

FIG. 14 shows results indicating that intravenous delivery of t-PA, but not DSPA, increases NMDA-induced striatal lesion volume. See paragraph [00100].

FIG. 15 shows results indicating that DSPA Injected Intravenously after NMDA-induced injury crosses the blood-brain-barrier. See paragraph [00100].

FIG. 16 shows results regarding patient population from DIAS and DEDAS clinical studies. See also paragraph [00119].

FIG. 17 describes the most relevant baseline data comparing all recruited patients so far. It is important to note that the 90 μg group seems to have more patients with a higher age (risk factor for bleeding) and that there seems to be a trend for later time to treatment. This is however true for comparing the “old” and “new” patients and is probably due to the fact the treatment window was changed during the first part of the DIAS trial. One symptomatic bleeding occurred in the 90 μg dose group so far and none in the 62.5 μg tier. The NIHSS (stroke severity) at entry is comparable to the 25 mg group. None of the differences between the groups are thought to have any statistical or medical relevance, since there seems to be a good overlap and spread amongst the groups. Note that the graphs represent the upper and lower quartiles. The median is outlined as a prominent line. In the original Figure, blue graphs represented data from the interim analysis, red represented the 62.5 μg tier and green the 90 μg tier. See also paragraph [00119].

FIG. 18 shows asymptomatic and symptomatic hemorrhagic transformation and describes the key issues of the safety aspects of the study (siCH). There has been one symptomatic bleeding in the 36 patients (62.5 and 90 μg/kg dose tiers) since the study has been restarted. In comparison to the previous bleedings several aspects are of interest. The bleeding occurred rather late (28 h after treatment), this seems to be the first patient that did not have any of the previous present risk factors (age, high glucose level, high NIHSS). The asymptomatic transformations seem to show no dose dependency, which is in line with a substantial fairly high incidence in untreated patients as is known from previous published trials. The patient who had siCH showed signs of reperfusion and, at the time of 4-8 h MRI, also signs of asymptomatic hemorrhagic transformation. See also paragraph [00119].

FIG. 19 describes vessel opening as measured by TIMI change. As has been stated by some of the investigators at the recent investigator meetings that MRA underrates the vessel opening. We will need to address this in the evaluation with the new TISS scale (this scale has been designed for the DIAS and DEDAS trial, representing a combination of TIMI and PWI changes, which has been adapted based on the data of the first 47 patients and is used in an exploratively manner). Reperfusion as assessed by PWI (FIG. 25 gives a slightly different picture). One has to keep in mind that the TIMI scale shows the reperfusion of larger vessels only, whereas PWI is a consecutive (i.e. later) change of blood flow into the penumbra, which also can reflect an improvement of the microcirculation. Thus it seems logical that the timelines and the “kinetics”/magnitude of these measures appear to follow slightly different algorithms. However one would have to be alarmed if they would show into different directions. In both modes the 90 μg group reaches values, which are similar to the 25 mg group, which was the best group of the “old” patients (i.e. the first unblinded data set of the interim analysis). It is difficult to assess if the “real” opening rate is eventually higher or a bit lower, but it is definitively better than the placebo group. For raw data see Tables 1 and 2 below. See also paragraph [00119].

FIG. 20 shows results regarding reperfusion as indicated by reduction on PWI. See also paragraph [00119].

FIG. 21 shows a sign of a bell shaped dose response curve for PWI but not for TIMI. Whereas a dose increase for TIMI seems to be there when the TIMI and TISS already show a bell shaped curve may be explained by the fact that TIMI just looks at (large) vessels, while PWI and TISS look at reperfusion effects on tissue. Whereas higher doses lead to more rapid and complete vessel opening this may lead also to some reperfusion damage which could be the reason for the bell shaped curve of the PWI and TISS results. See also paragraph [00119].

FIG. 22 shows NIHSS improvement 4-8 h after treatment and day 7 and how the reperfusion translates into the acute clinical improvement of the stroke patients measured as improvement of NIHSS stroke scale. It is amazing that both the 62.5 μg/kg and the 90 μg/kg do better than any dose group at 4-8 h after medication. One might speculate that this is an indicator that reperfusion injury occurred in the previous higher doses. This trend is continued up to day 7, where both dose groups do better than the best of the previous groups (25 mg) both when bleedings are included or excluded in the analysis. An important point is that the 62.5 μg/kg dose group is as good as the 25 mg and the 90 μg/kg is better than the 25 mg at day 7. See also paragraph [00119].

FIG. 23 shows hypothetical dose response curves for Desmoteplase in stroke patients. See also paragraph [00119].

FIG. 24 (Table 4) shows the raw blinded data for the 62.5 μg/kg dose tier. See also paragraph [00119].

FIG. 25 (Table 5) shows the raw blinded data for the 90 μg/kg dose tier. See also paragraph [00119].

FIG. 26 (Table 6) shows the baseline characteristics of patients (ITT population). See also paragraph [00197].

FIG. 27 (Table 7) shows safety data (ITT population). See also paragraph [00198].

FIG. 28 (Table 8) shows efficacy of treatment. See also paragraph [00199].

FIG. 29 (Table 9) shows the baseline characteristics of patients treated >6-9 h after onset of stroke. See also paragraph [00200].

FIG. 30 (Table 9) shows efficacy in patients treated >6-9 h after onset of stroke. See also paragraph [00201].

FIG. 31 (Table 9) shows univariate odds ratios over placebo regarding reperfusion (gray bars) and good clinical outcome. See also paragraph [00202].

### NON-NEUROTOXIC PLASMINOGEN ACTIVATORS

Plasminogen activators for the treatment of stroke, the enzymatic activity of which is highly selectively increased by fibrin many times over, i.e. by a factor greater than 650, are known from the international patent application PCT/EP02/12204.

The character and administration of these plasminogen activators are based on the following observations. The neurotoxicity of the tissue-plasminogen activator (t-PA) is mainly due to the fact that the blood-brain barrier is impaired or breaks down as a consequence of the tissue destruction in the brain caused by the stroke. Because of these circumstances, the fibrinogen circulating in the blood can permeate into the neural tissue of the brain. There, the fibrinogen activates t-PA, which indirectly leads to further tissue damage by activating the glutamate receptor or by the activation of plasminogen. (see above).

In order to prevent this effect, the administration of a plasminogen activator that displays an increased selectivity for fibrin and, therefore, a decreased potential of activation by fibrinogen, has been suggested. This has the consequence that these plasminogen activators will not be activated or, in comparison to t-PA, will be activated to a much lower extent when fibrinogen permeates from the blood into the neural tissue in consequence of the damaged blood-brain barrier, since their activator fibrin cannot enter the neural tissue for reason of its size and insolubility. These plasminogen activators are thus non-neurotoxic.

a) Genetically Modified Plasminogen Activators

According to one embodiment of the invention, non-toxic plasminogen activators are used, which comprise at least one element of the so called cymogene triade. A comparable triade is known from the catalytic center of serine proteases of the chymotrypsine family consisting of three interacting amino acids aspartate 194, histidine 40 and serine 32. However, this triade does not exist in t-PA, which belongs also to the family of chymotrypsine like serine proteases. Nevertheless, it is known, that the directed mutagenesis of native t-PA for the purpose of introducing at least one of the above amino acids at a suitable position results in a reduced activity of the pro-enzyme (single chain t-PA) and to an increased activity of the mature enzyme (double chain t-PA) in the presence of fibrin. Therefore, the introduction of at least one amino acid of the triade—or of an amino acid with the respective function in the triade—can increase the cymogenity of t-PA (i.e., the ratio between the activity of the mature enzyme an the activity of the pro-enzyme). As a result, the fibrin specificity is remarkably increased. This is due to conformational interaction between the introduced amino acid residue and/or amino acid residues of the wild type sequence.

It is known that the mutagenesis of the native t-PA with substitution of Phe305 by H is (F305H) and of Ala 292 by Ser (A292S) leads to a 20-fold increase of the cymogenity, whereas the variant F305H alone already leads to 5 times higher cymogenity (E L Madison, Kobe A, Gething M-J; Sambrook J F, Goldsmith E J 1993: Converting Tissue Plasminogen Activator to a Zymogen: A regulatory Triad of Asp-His-Ser; Science: 262, 419-421). In the presence of fibrin these t-PA mutants show an activity increase of 30,000 times (F305H) and 130,000 times (F305H, A292S) respectively. In addition, these mutants comprise a substitution of Arg275 to R275E in order to prevent cleavage by plasmin at the cleavage site Aug275-Ile276, thereby converting the single chain t-PA to the double chain form. The mutant site R275E alone leads to a 6.900 fold increase of the fibrin specificity of t-PA (K Tachias, Madison EL 1995: Variants of Tissue-type Plasminogen Activator Which Display Substantially Enhanced Stimulation by Fibrin, in: Journal of Biological Chemistry 270, 31: 18319-18322).

The positions 305 and 292 of t-PA are homologous to the positions His40 and Ser32 of the known triade of the chymotryptic serine proteases. By the corresponding substitutions introducing histidine or respectively serine, these amino acids can interact with the aspartate477 of t-PA resulting in a functional triade in the t-PA mutants (Madison et al., 1993).

These t-PA mutants can be used for the treatment of stroke according to the invention because they show no or—compared to wild type t-PA—a significantly reduced neurotoxicity due to their increased fibrin specificity. For the purpose of disclosure of the mentioned t-PA mutants F305H; F305H; A292S alone or in combination with R275E we incorporate hereby the publications of Madison et al., (1993) and Tachias and Madison (1995) fully by reference.

The increase of fibrin specificity of plasminogen activators can alternatively be achieved by a point mutation of Asp194 (or an aspartate at a homologous position). Plasminogen activators belong to the group of serine proteases of the chymotrypsin family and therefore comprise the conserved amino acid Asp194, which is responsible for the stability of the catalytic active conformation of the mature proteases. It is known that Asp194 interacts with His40 in the cymogenic form of serine proteases. After the cymogene is activated by cleavage this specific interaction is interrupted and the side chain of the Asp194 rotates about 170° in order to form a new salt bridge with Ile16. This salt bridge essentially contributes to the stability of the oxyanione pocket of the catalytic center of the mature serine proteases. It is also present in t-PA.

The introduction of a point mutation replacing Asp194 prima facie impedes the formation, or the stability of, the catalytic confirmation of serine proteases. Despite this, the mutated plasminogen activators show a significant increase of activity in the presence of their co-factor fibrin—especially in comparison to the mature wild type form—which can only be explained in a way that the interaction with fibrin allows a conformational change promoting catalytic activity (L Strandberg, Madison E L, 1995: Variants of Tissue-type Plasminogen Activator with Substantially Enhanced Response and Selectivity towards Fibrin co-factors, in: Journal of Biological Chemistry 270, 40: 2344-2349).

In conclusion, the Asp194 mutants of the plasminogen activators show a high increase of activity in presence of fibrin, which enables their use according to the invention.

In another embodiment according to the invention, a mutant t-PA is used, in which Asp194 is substituted by glutamate (D194E) or respectively by asparagine (D194N). In these mutants the activity of t-PA is reduced 1 to 2000 fold in the absence of fibrin, whereas in the presence of fibrin, an increase of activity by a factor of 498,000 to 1,050,000 can be achieved. These mutants can further comprise a substitution of Arg15 to R15E, which prevents the cleavage of the single chain t-PA at the peptide bond Arg15-Ile16 by plasmin, leading to the double chain form of t-PA. This mutation alone increases the activation of t-PA by fibrin by the factor 12,000. For reasons of disclosure of the t-PA mutations at positions 194 and 15, the publications of Strandberg and Madison (1995) are hereby fully incorporated by reference.

An increase of the fibrin dependency of plasminogen activators can also be achieved by the introduction of point mutations in the so called “autolysis loop”. This element is known from trypsine; it can also be found as a homologous part in serine proteases and is characterized by three hydrophobic amino acids (Leu, Pro and Phe). The autolysis loop in plasminogen activators is responsible for the interaction with plasminogen. Point mutations in this area can have the effect that the protein-protein interaction between plasminogen and plasminogen activators cannot be effectively formed any longer. These mutations are only functionally relevant in the absence of fibrin. In the presence of fibrin, they, in contrast, are responsible for an increased activity of the plasminogen activators (K Song-Hua, Tachias K, Lamba D, Bode W, Madison E L, 1997: Identification of a Hydrophobic exocite on Tissue Type Plasminogen Activator That Modulates Specificity for Plasminogen, in: Journal of Biological Chemistry 272; 3, 1811-1816).

In another embodiment, t-PA is used showing point mutations in the positions 420 to 423. If these residues are substituted by directed mutagenesis this increases the fibrin dependency of t-PA is increased by a factor up to 61,000 (K Song-Hua et al.). Song-Hua et al. examined the point mutations L420A, L420E, S421G, S421E, P422A, P422G, P422E, F423A and F423E. These publications are hereby fully incorporated by reference.

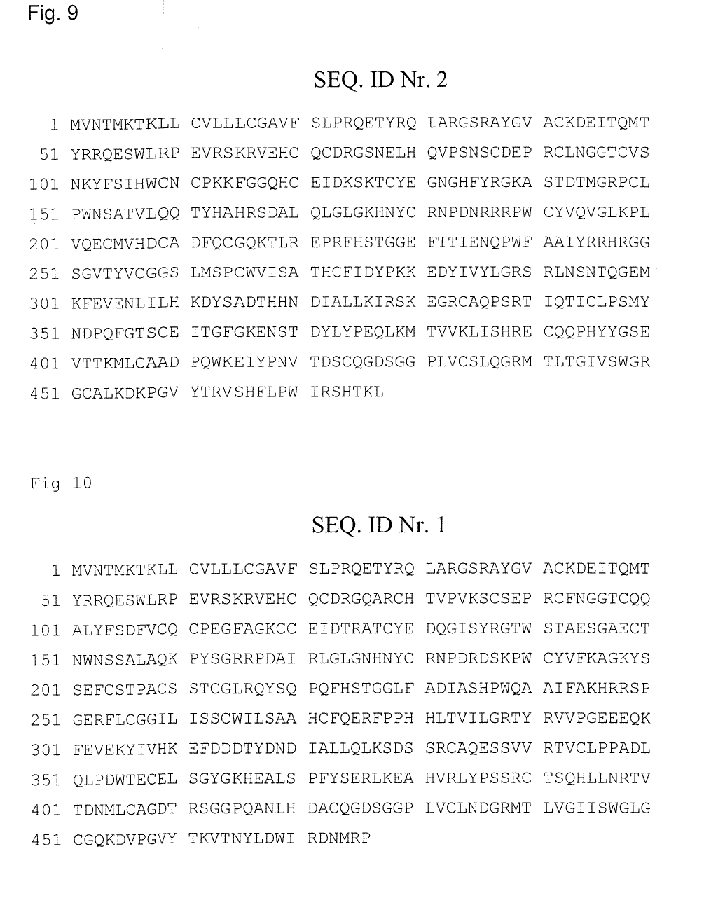

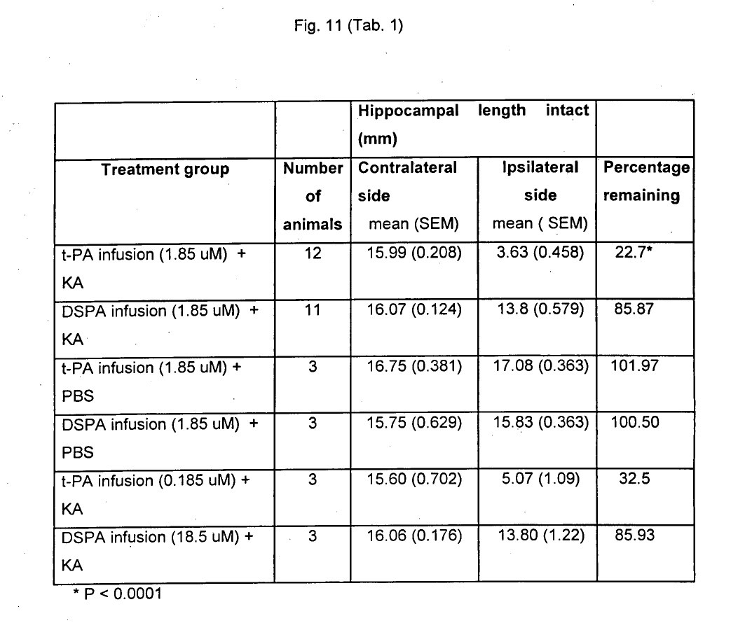

According to a further embodiment, a modified tissue plasminogen activator with an amino acid sequence according to SEQ ID No. 1 (FIG. 10) is used. This modified t-PA differs from the wild type t-PA by the exchange of the hydrophobic amino acids in the position 420 to 423 in the autolysis loop as follows: His420, Asp421, Ala422 and Cys423. In one embodiment of the invention, this t-PA contains a phenyl alanine at the position 194. Further the position 275 can be occupied by glutamate. Other t-PAs can also have the position 194 occupied by phenyl alanine.

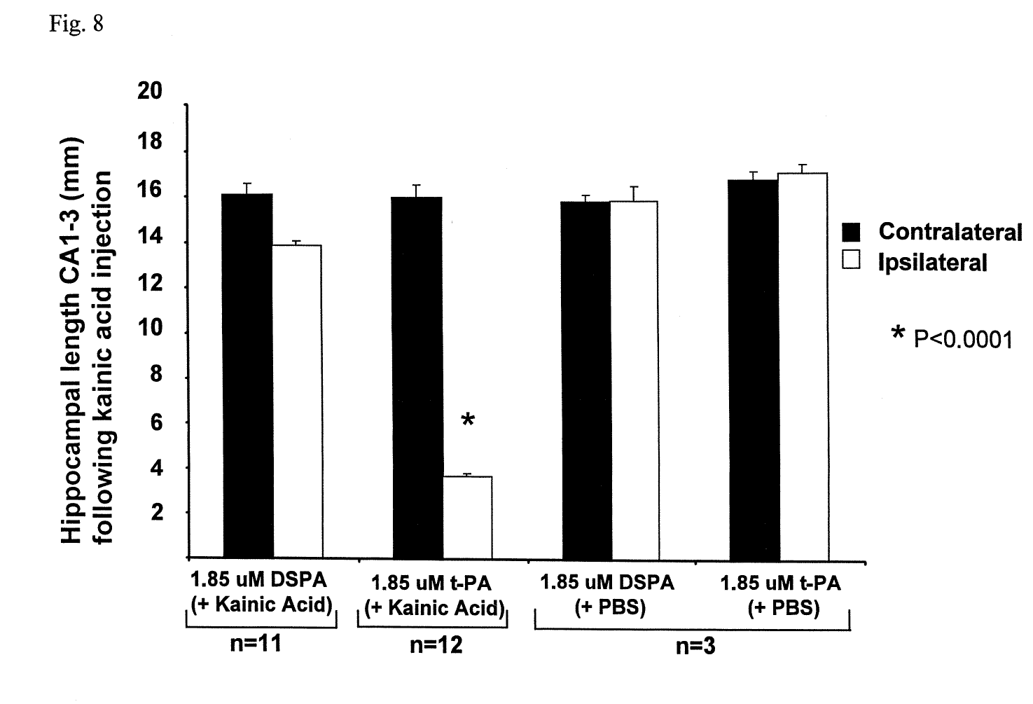

Further, a modified urokinase can be used according to the invention. The urokinase according to the invention can comprise the amino acid sequence according to SEQ ID No. 2 (FIG. 9) in which the hydrophobic amino acids of the autolysis loop are substituted by Val420, Thr421, Asp422 and Ser423. In one embodiment of the invention, the urokinase is carrying an Ile275 and a Glu194. This mutant shows—in comparison to wild type urokinase—a 500-fold increased fibrin specificity.

Both mutants—urokinase as well as t-PA—were analyzed in semi quantitative tests and showed a increased fibrin specificity in comparison to the wild type t-PA.

b) plasminogen Activator from Desmodus rotundus (DSPA)

The plasminogen activator (DSPA) from the saliva of the vampire bat (Desmodus rotundus) also shows a highly increased activity in the presence of fibrin—in specific a 100,000-fold increase. Thus, it can also be used according to the invention. The term DSPA comprises four different proteases, which fulfill an essential function for the vampire bat, namely an increased duration of bleeding of the wounds of pray (Cartwright, 1974). These four proteases (DSPAα1, DSPAα2, DSPAβ, DSPAγ) display a high similarity (homology) to each other and to the human t-PA. They also show similar physiological activities, leading to a common classification under the generic term DSPA. DSPA is disclosed in the patents EP 0 352 119 A1 and of U.S. Pat. Nos. 6,008,019 and 5,830,849 which are hereby fully incorporated by reference.

DSPAα1 so far is the best analyzed protease from this group. It has an amino acid sequence with a homology greater than 72% in comparison to the known human t-PA amino acid sequence (Krätzschmar et al, 1991). However, there are two essential differences between t-PA and DSPA. Firstly, all DSPAs have full protease activity as a single chain molecule, since it is—in contrast to t-PA—not converted into a double chain form (Gardell et al., 1989; Kratzschmar et al., 1991). Secondly, the catalytic activity of DSPA is nearly absolutely dependent on fibrin (Gardell et al., 1989; Bringmann et al., 1995; Toschie et al., 1998). For example the activity of DSPAα1 is increased 100,000 fold in the presence of fibrin whereas the t-PA activity is only increased 550 fold. In contrast, DSPA activity is considerably less strongly induced by fibrinogen, since it only shows a 7 to 9 fold increase (Bringmann et al., 1995). In conclusion, DSPA is considerably more dependent of fibrin and much more fibrin specific as wild type t-PA, which is only activated 550-fold by fibrin.

Because of its fibrinolytic characteristics and the strong similarity to t-PA, DSPA is an interesting candidate for the development of a thrombolytic agent. Despite this, the therapeutic use of DSPA as a thrombolytic agent was restricted to the treatment of myocardinal infarction in the past, because—due to the contribution of t-PA to the glutamate induced neurotoxicity—no justified hopes existed that a plasminogen activator related to t-PA could reasonably be used for a treatment of acute stroke.

Surprisingly, we have shown that DSPA has no neurotoxic effects even though it shows a high resemblance (homology) to t-PA and even though the physiological effects of the molecules are comparable to a large extent. The above conclusion led to the idea that DSPA after all may be successfully used as a thrombolytic agent for the therapy of stroke without causing severe risks of neuronal tissue damage. We discovered that DSPA can also be used later than 3 hours after the onset of stroke symptoms.

Experimental Proof for the Missing Neurotoxicity of DSPA

The new teaching is based on in vivo comparative examinations of the neurodegenerative effect of t-PA on one side and of DSPA on the other side, which are performed by using the so called kainic acid model and a model for the examination of NMDA induced lesion of the striatum.

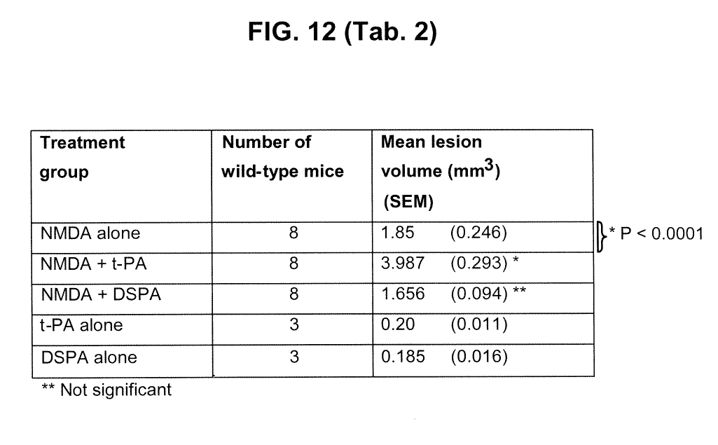

The kainic acid model (also kainic acid injury model) is based on the stimulation of the neurotoxic glutamate cascade by the external application of kainic acid (KA) as an agonist of the glutamate receptor of the kainic acid type (KA type) and of the NMDA and AMPA glutamate receptors. Using a t-PA deficient mouse stem as an experimental model it was possible to show that the sensitivity of the laboratory animals against kainic acid only reached the level of wild type mice after a supplementary application of external t-PA. In contrast, an infusion of an equimolar concentration of DSPA under the same experimental conditions does not restore the sensitivity to kainic acid (KA). It was concluded that the neurotoxic effect of t-PA was not induced by DSPA. A summary of these results is shown in FIG. 12 (Table 2).

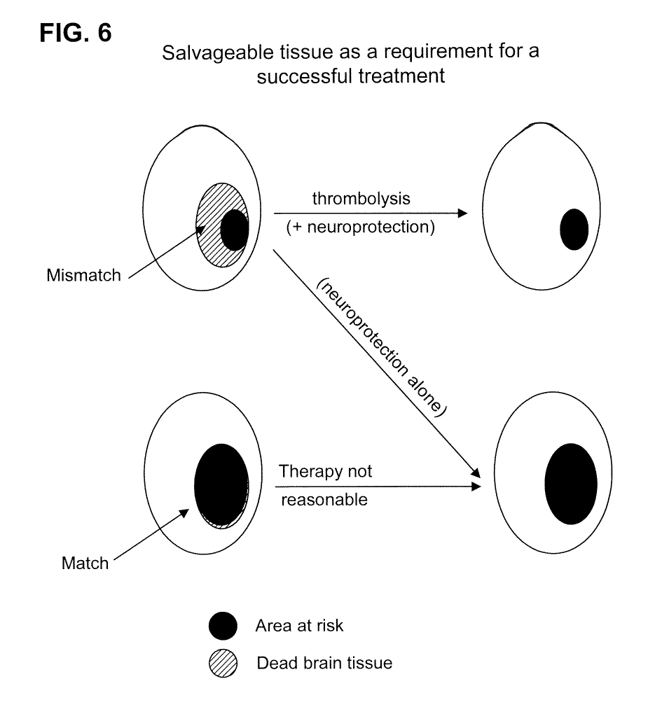

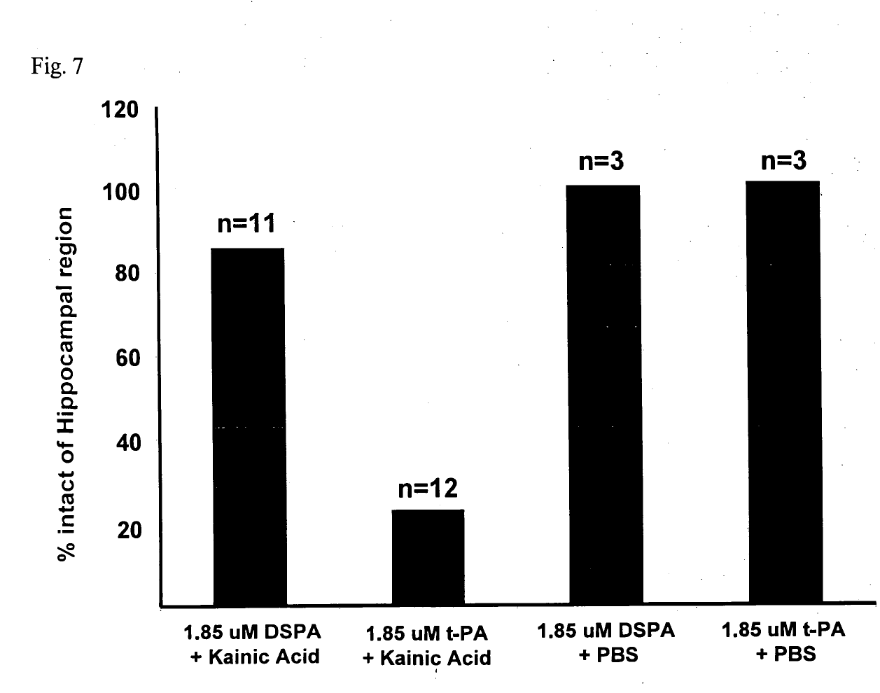

Quantitative examinations based on this model revealed that even a 10-fold increase of the DSPA concentration could not restore the sensitivity of the t-PA deficient mice to the KA treatment whereas already a 10-fold lower t-PA concentration led to KA induced tissue damages. This leads to the conclusion that DSPA possesses an at least 100 fold lower neurotoxic potential as t-PA with respect to the stimulation of the neurodegeneration after KA treatment (see also FIGS. 7 and 8).

In the second model of neurodegeneration, the possible effects of t-PA as well as DSPA on the stimulation of the NMDA dependent neurodegeneration were compared to wild type mice. For this purpose, NMDA (as an agonist of the glutamate receptor of the NMDA type) was injected in wild type mice alone or in combination with either t-PA or DSPA. This model allows the comparison of the effects of these proteases under conditions, which always lead to a neurodegeneration and to an influx of plasma proteins due to the breake down of the blood brain barrier (Chen et al., 1999).

While working on this model the injection of NMDA led to reproducible lesions in the striatum of mice. The volume of lesions was increased by a combined injection of t-PA and NMDA by at least 50%. The co-injection with DSPAα1, in contrast, did not lead to an increase or extension of the lesions caused by NMDA. Even in the presence of plasma proteins, which can freely diffuse in the region of the lesion induced by NMDA, DSPA did not result in an increase neurodegeneration. A summary of these results is given in FIG. 12 (Table 2).

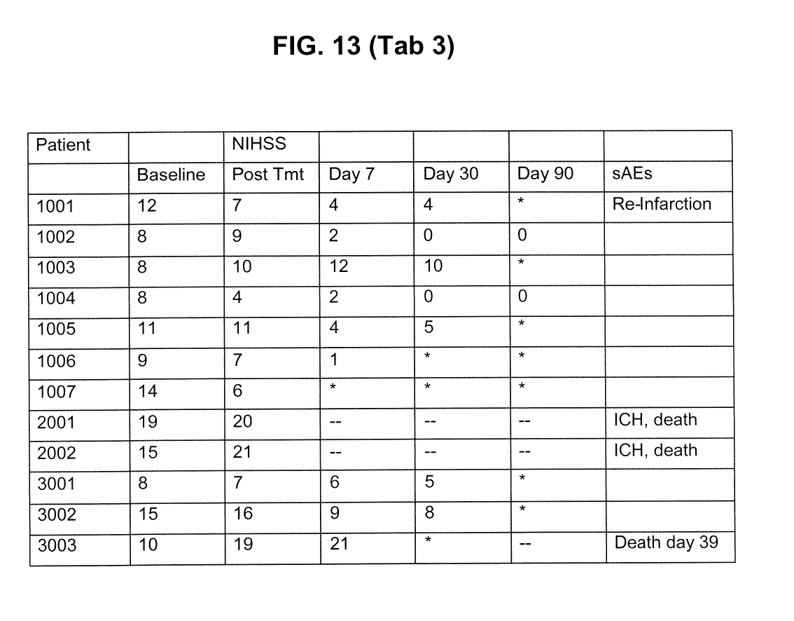

First results from clinical trials show the transferability of these results also for the treatment of stroke in humans. It was found that significant improvements can be achieved in patients after a successful perfusion (improvement by 8 points NIHSS or NIHSS score 0 to 1). This is shown in FIG. 13 (Table 3).

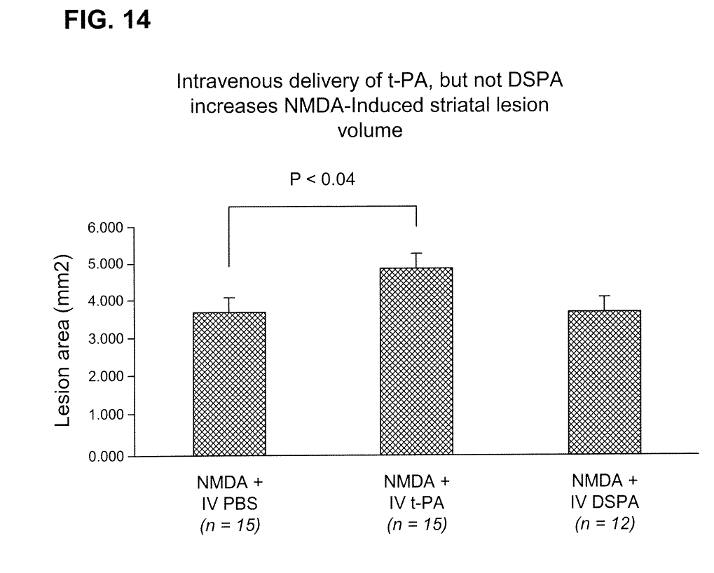

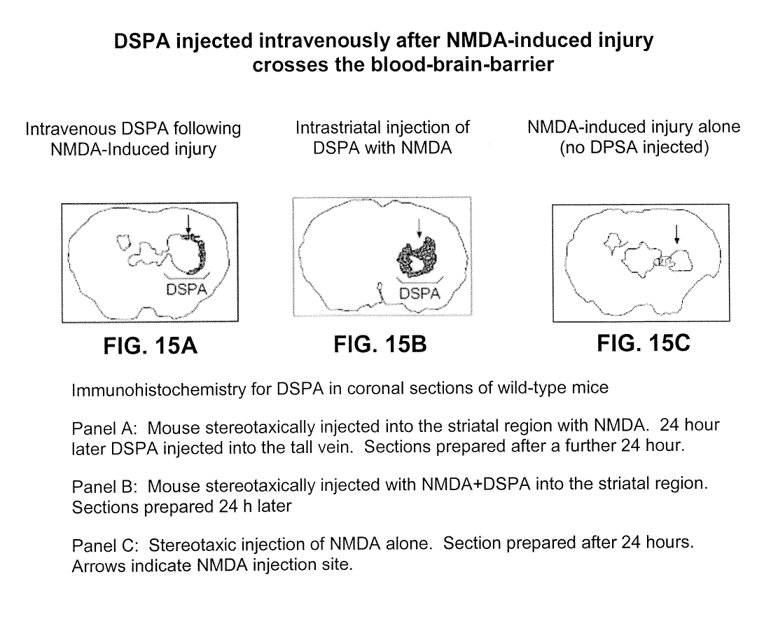

In a further experiment, it was tested whether t-PA and DSPA were able to permeate the damaged blood-brain barrier when being intravenously administered and in consequence increase the tissue lesions in the brain. To address this question, mice were stereotactically injected with NMDA in order to produce tissue lesions in the striatum, followed by an intravenous application of t-PA or DSPA 6 or 24 h after the NMDA injection. In comparison to a negative control, the experimental animals showed an increase of the NMDA induced damaged tissue area of about 30% when t-PA was given as an infusion 24 hours after the NMDA injection; in contrast, the same treatment with DSPA did not produce such an increase of tissue damage, although its penetration into the damaged tissue area was positively detected by means of an antibody staining (see FIGS. 14 and 15). When t-PA or DSPA were intravenously applied in a corresponding manner 6 hours after the NMDA-injection, an increase of the damaged tissue area was not detected. This may be explainedif the blood-brain barrier still provided a sufficient barrier function at the moment of the t-PA or DSPA injection.

These results show, that DSPA constitutes a mostly inert protease in the central nervous system of a mammal (and thus also of a human) and, in contrast to t-PA, does not produce a potentiation of the neurotoxicity induced by KA or NMDA. This lack of neurotoxicity—against the general expectation—makes DSPA a suitable thrombolytic for the treatment of acute stroke.

Therapeutic Potential of the Non-Neurotoxic Plasminogen Activators

We have found that the lacking neurotoxicity of DSPA and of other non-neurotoxic plasminogen activators (see above) offer the advantage in stroke treatment that the use of these plasminogen activators—in contrast to the wild type t-PA—is not limited to a short maximum period of only 3 hours after the onset of stroke. On the contrary, the treatment can be initiated later—for example after 6 hours or even later, since there is nearly no risk of stimulating excitotoxic responses. First clinical trials with DSPA prove a safe treatment of patients even in a time range of over 6 to 9 hours after the onset of stroke symptoms.

This option of a timely unlimited treatment with non-neurotoxic activators is useful, since it allows for the first time to treat patients with acute stroke symptoms safely even when diagnosis is delayed or the onset of the stroke cannot be determined with sufficient security. In the prior art, this group of patients was excluded from thrombolytic therapy with plasminogen activators due to unfavorable risk estimation. Consequently, an essential contra-indication for the authorized use of a thrombolytic agent for stroke is eliminated.

Application of Plasminogen Activators

In contrast to the already established stroke therapeutic rt-PA, no validated information is yet available for a possible mode of application for the stroke treatment with the non-neurotoxic plasminogen activators.

One embodiment of the invention provides an advantageous mode of administration for these non-neurotoxic plasminogen activators.

According to another embodiment of the invention, plasminogen activators, the activity of which is increased in the presence of fibrin by a factor greater than 650, are intravenously applied for the treatment of stroke.

The intravenous administration of these non-neurotoxic plasminogen activators for the treatment of stroke has already been evaluated in clinical trials in which DSPA—as an example for this group of fibrinolytics—was intravenously applied in these patients, thereby causing only minor side effects.

These results of the clinical studies were unexpected, since it was well known, that the intravenous application of t-PA and other common fibrinolytics is associated with a severe risk of cerebral bleedings (see above).

In order to reduce intra-cerebral bleedings, recent efforts were made to develop strategies to apply these substances no more intravenously, but via the intra-arterial route into the direct vicinity to the intravascular thrombus by means of a catheter. Practical experience for this is already available for the recombinantly produced urokinase (PROKAT as a study with pro-urokinase). Since this mode of application allows for a significant reduction of the total dose, a decrease in the dose-dependent side effects—thus also of the cerebral bleedings—is realised.

In general, these advantages of intra-arterial application however could be contravened by two possible drawbacks. First, this treatment prerequisites a time consuming preparation of the patient, which in stroke treatment is not possible to be realized within the given window in time of just 3 hours. On the one hand one sure can achieve a lower total dose. A higher concentration of the drug however will locally reach the terminal arterial vessels. In consequence of the impaired barrier function of the vascular endothelium in case of a stroke, the pharmaceutical substance will also reach the surrounding tissues in locally high concentrations. There it may then exert undesired side effects.

In contrast thereto, the concentration of the pharmaceutical substance will be diluted by the venous blood flow in case of an intravenous application. Thus, the intra-arterial injection is problematic in case of drugs having a tissue destructing potential like t-PA (Forth, Henschler, Rummel, Starke: “Pharmakologie und Toxikologie”, 6^{th} Edition, 1992, page 29).

However, the limitations complicating the intra-arterial application—the narrow window in time and the tissue damaging side effects—are not valid for the plasminogen activators applied according to the invention. Thus, the intra-arterial application constitutes a mode of application for non-neurotoxic plasminogen activators according to the invention.

Nevertheless, the applicants, also for the non-neurotoxic plasminogen activators, have also chosen to rely on intravenous application, which then surprisingly has proven to be effective.

In one embodiment, the mode of application according to the invention departs from the common therapeutic practice to mostly use an intramuscular injection or an intravenous drip infusion in order to administer proteins with an increased immunogenic potential, which has the aim to reduce the risk of an anaphylactic shock (“Gifttiere” v. Dietrich Mebs Wissenschaftliche Verlagsgesellschaft:, 2th Edition, 2000).

In contrast to the t-PA as a natural bodily substance, the plasminogen activators applied according to the invention are either foreign proteins derived from animals (like, e.g., DSPA) or genetically modified bodily proteins displaying novel epitopes in consequence of their structural differences. The potential accompanying problem of anaphylactic reactions—in particular when applying high therapeutic doses, like it is commonly necessary in case of intravenous application—applies also for other fibrinolytics consisting of foreign proteins like e.g. streptokinase.

In another embodiment, the plasminogen activators applied according to the invention are administered by means of a bolus injection (intravenous quick injection), which can also be administered as a single intravenous quick injection containing the complete therapeutic dose.

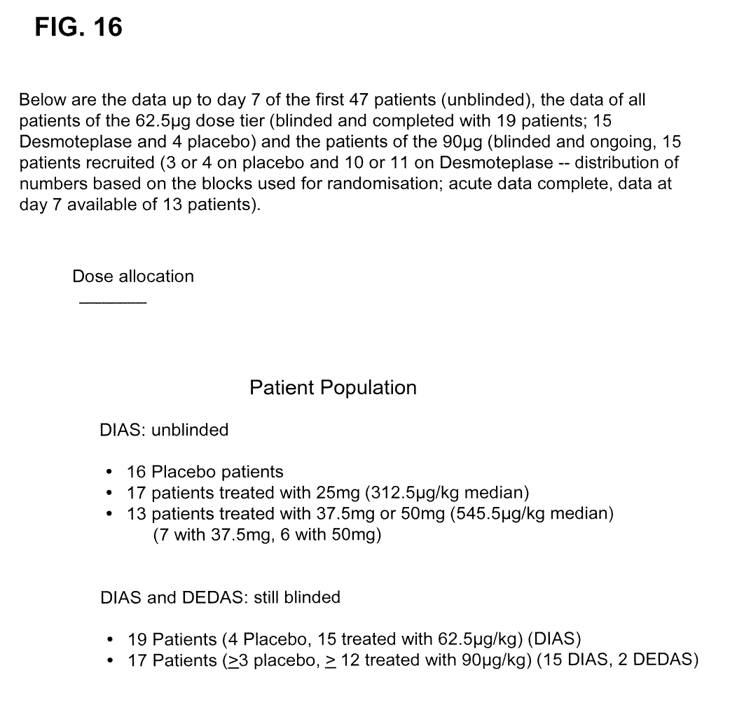

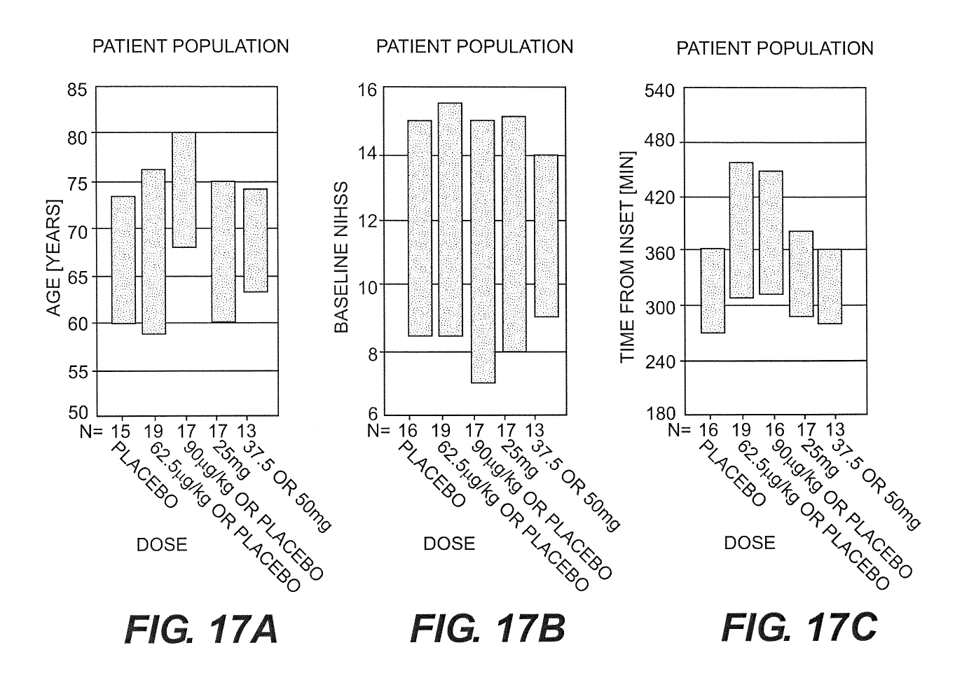

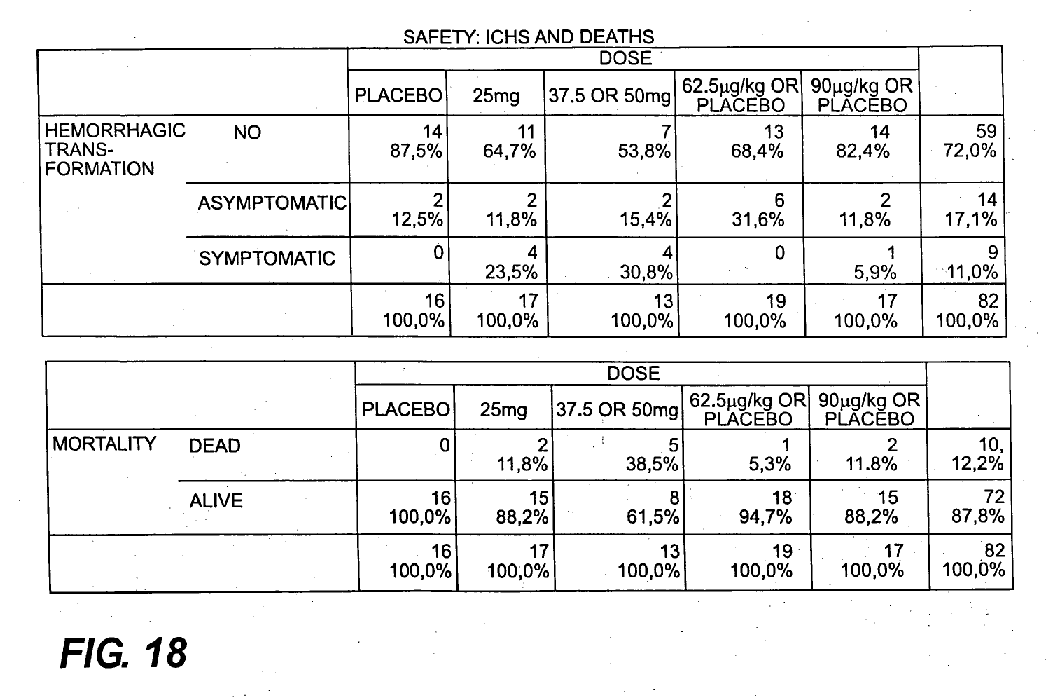

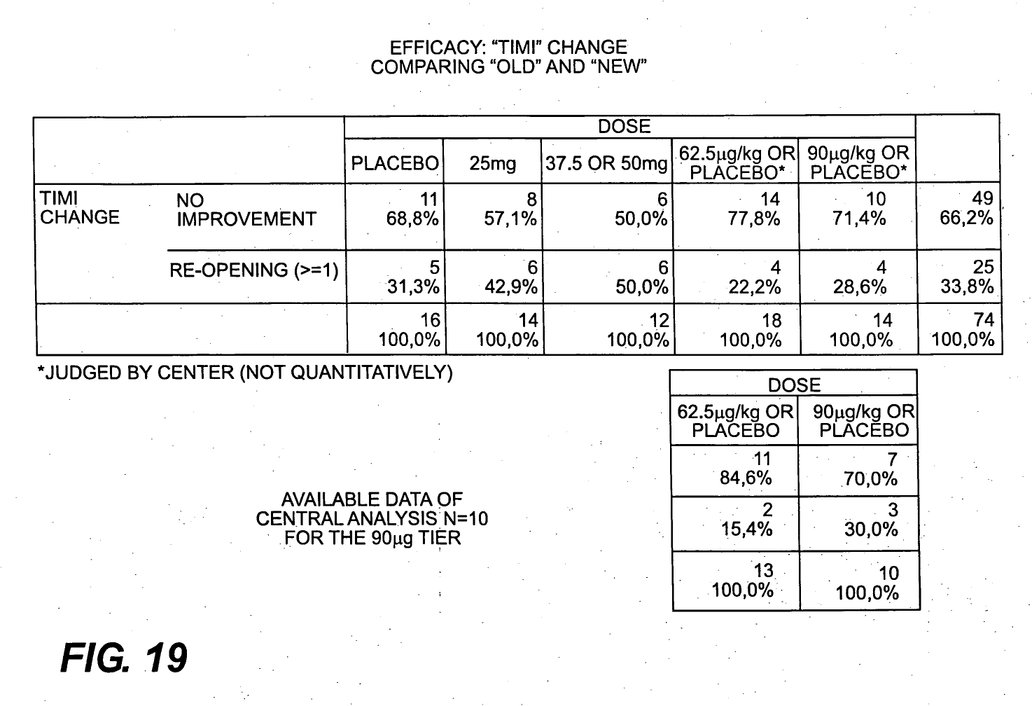

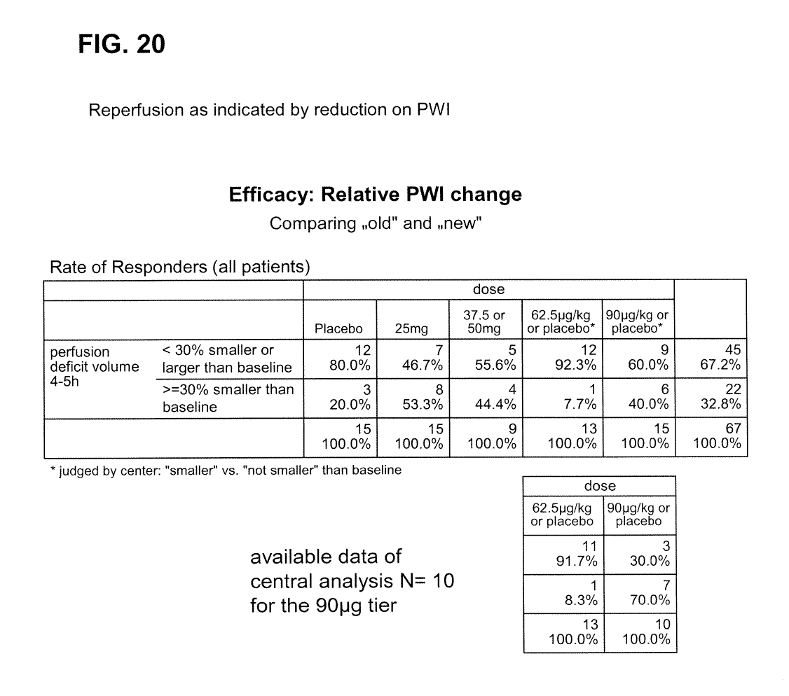

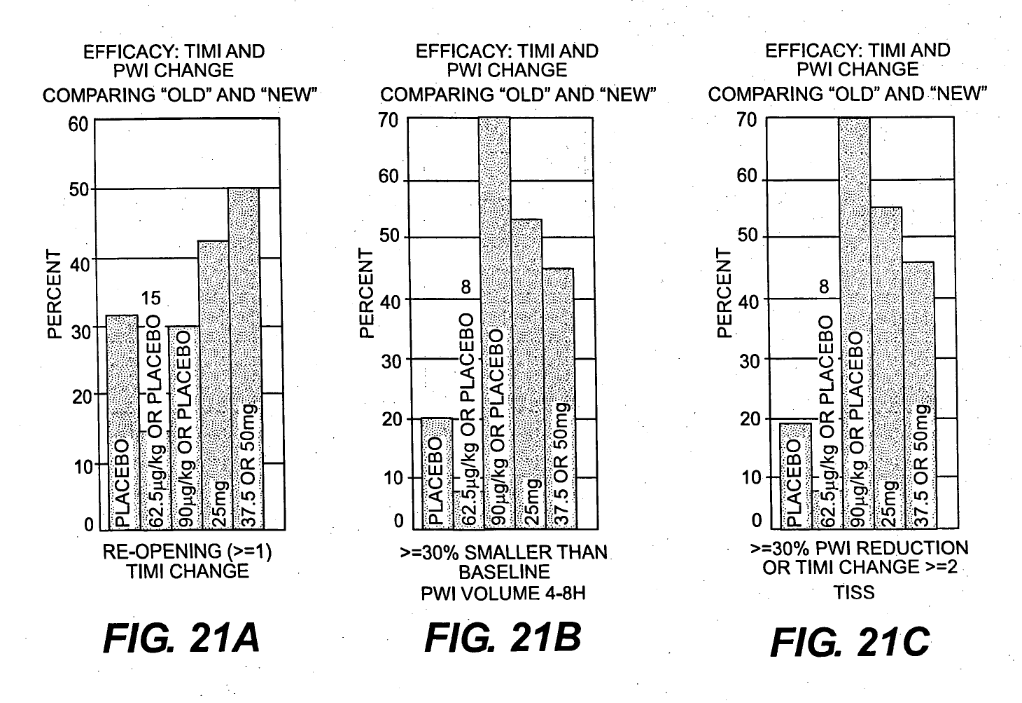

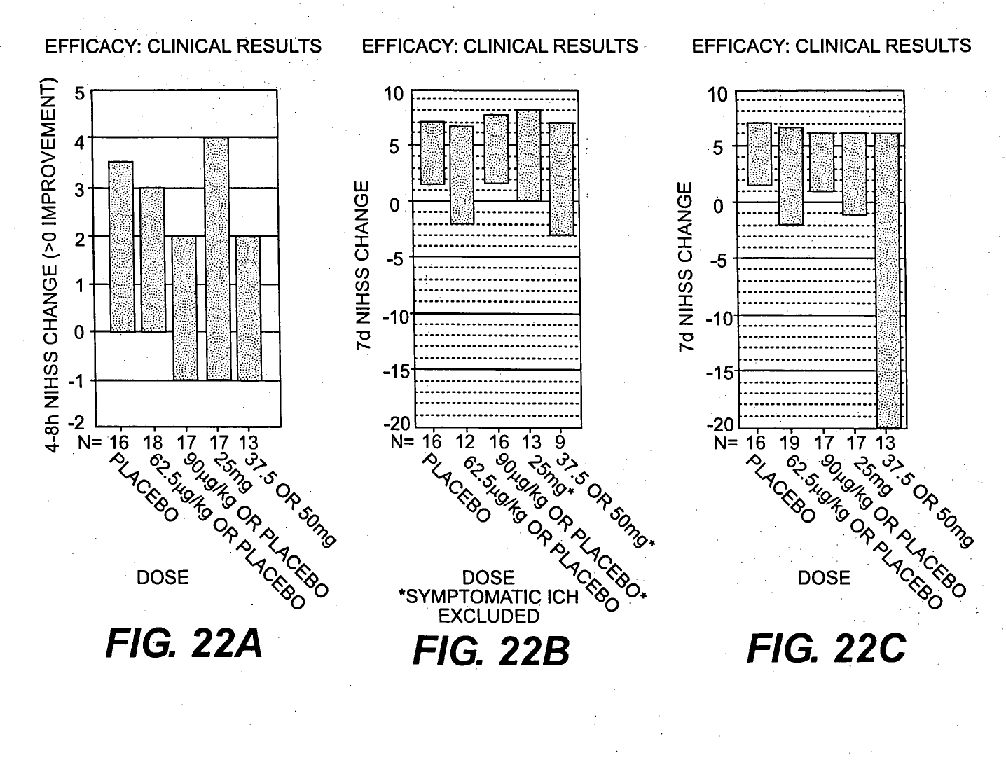

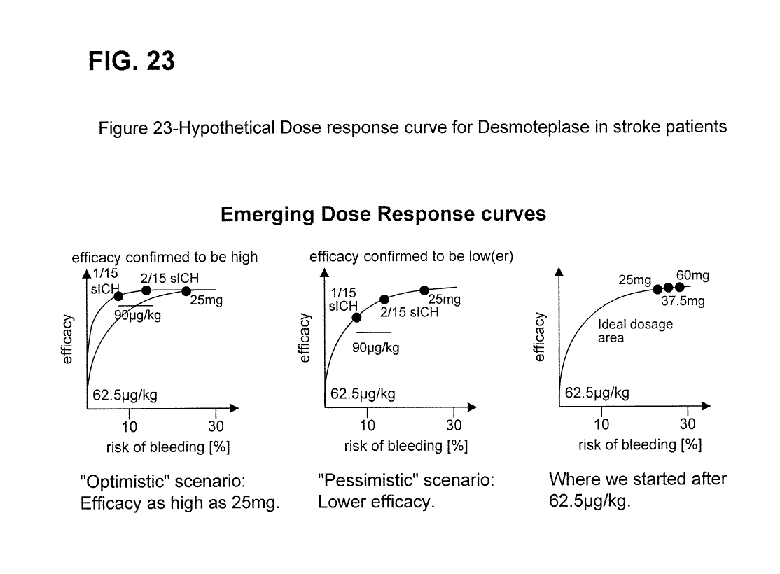

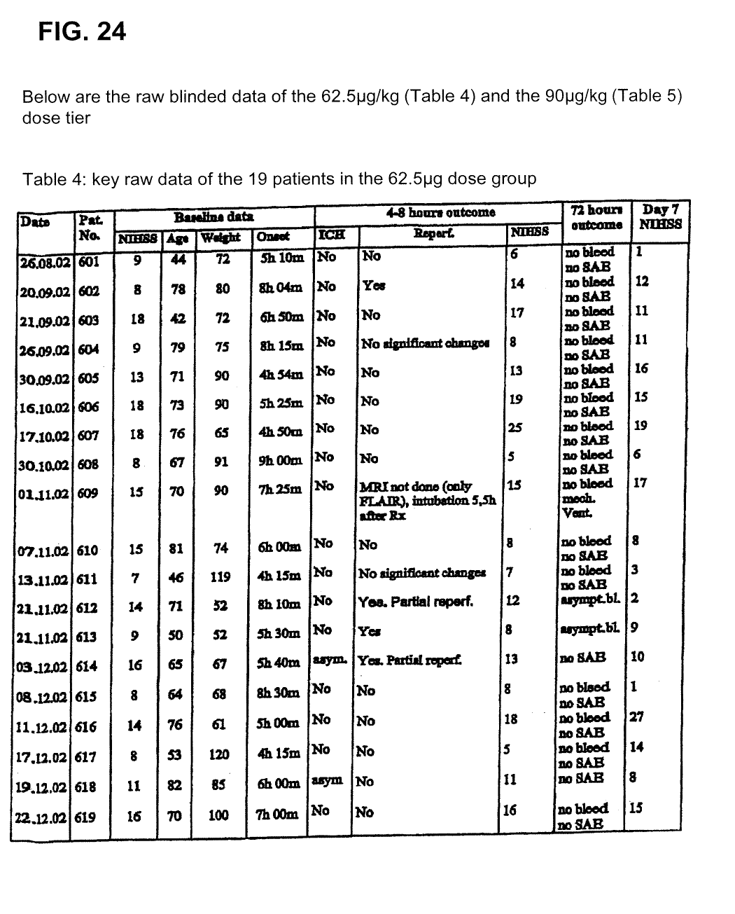

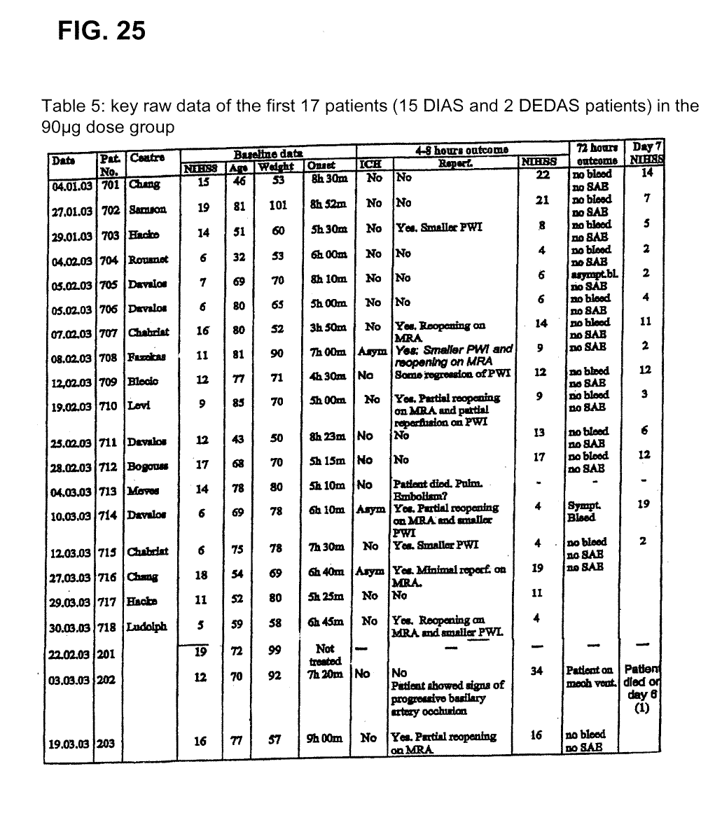

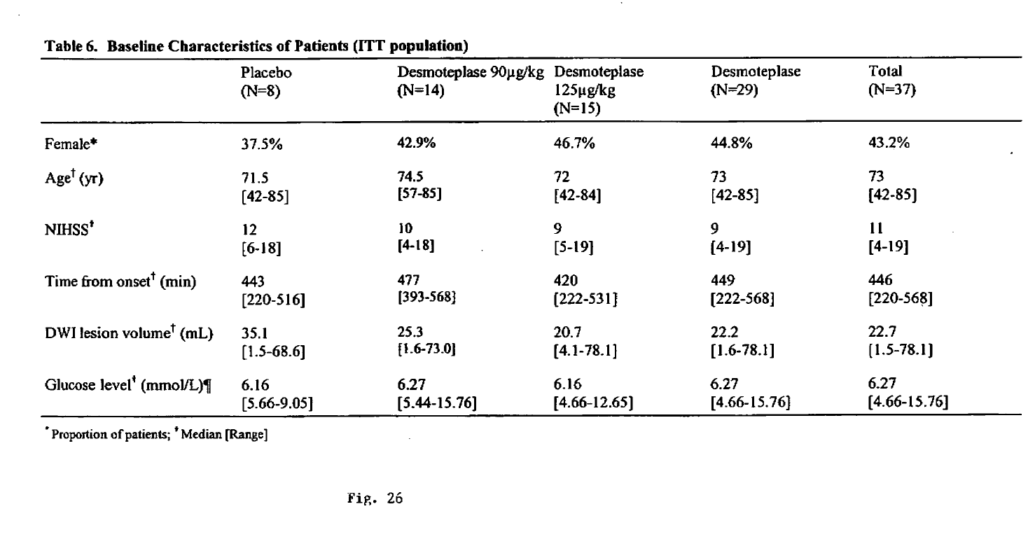

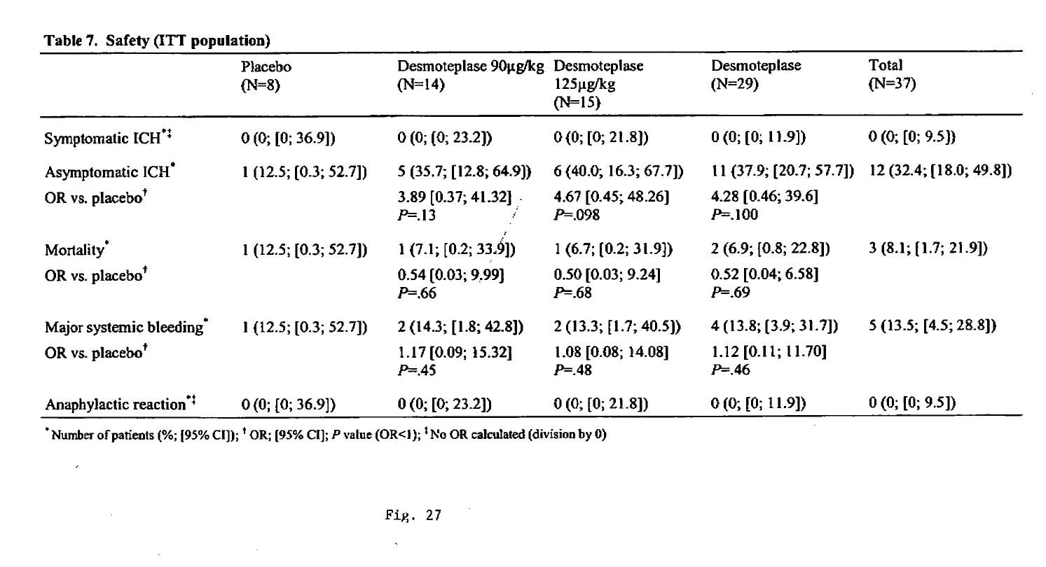

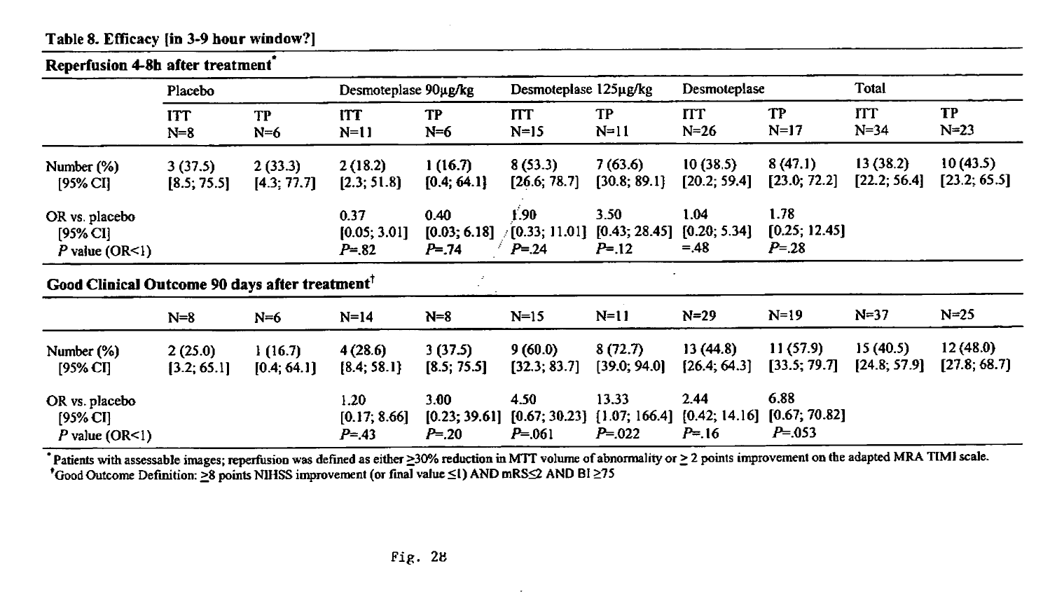

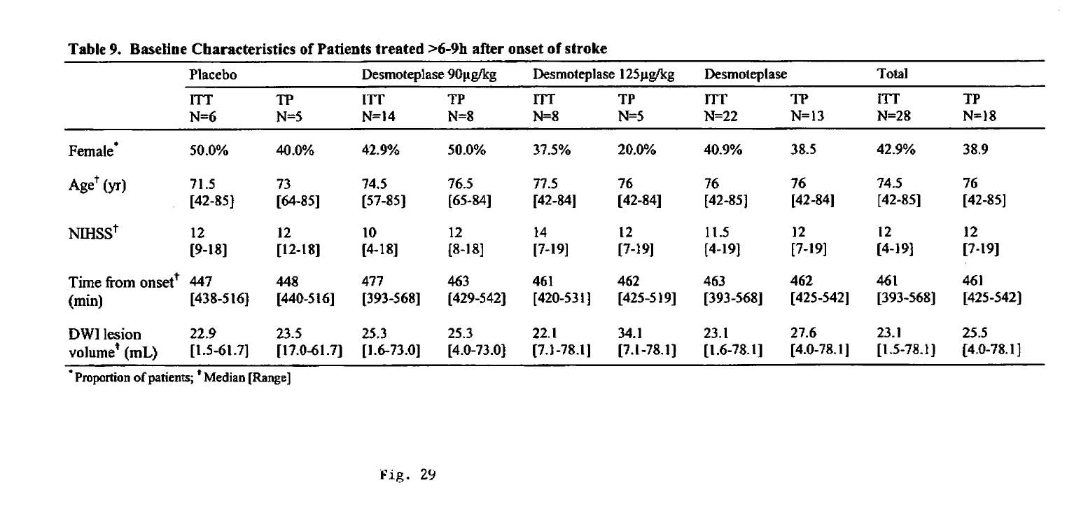

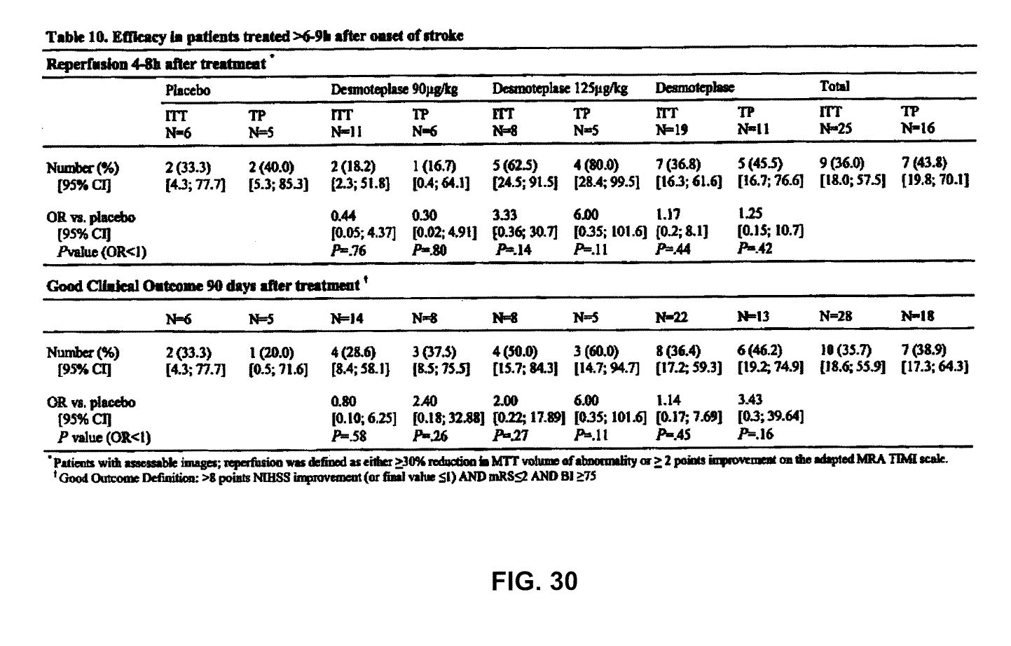

In the context of clinical studies it was found, that even the intravenous application of surprisingly low therapeutic doses lead to therapeutic results. Therapeutic results were achieved, e.g., with doses between 90 μg/kg and 230 μg/kg. Also, therapeutic results here were achieved with doses between 62.5 to 90 μg/kg. Therefore, a range of dosages with therapeutic effects includes 62.5 to 230 μg/kg. In the examined patients, the periods between the stroke and the application of the drug included periods between 3 and 9 hours. By means of suitable examination methods it was possible to determine the onset of a therapeutic effect (see FIGS. 16 to 25). We have also found therapeutic effects by the administration of non-neurotoxic plasminogen activators at a dose of 125 μg/kg.

Another embodiment of the invention is directed to the administration of non-neurotoxic plasminogen activators, such as, e.g., the DSPA variants, in doses between 60 and 130 μg/kg. Other doses that can be used in the methods of the invention include dose between 90 and 130 μg/kg. The present invention also contemplates dosages falling within a range defined by any two dose values recited in this application. As an exemplary illustration, the following dosages are within the scope fo the invention: dosages between 60 and 125 μg/kg, dosages between 125 and 230 μg/kg, and dosages between 62.5 and 130 μg/kg.

In one embodiment of the invention, non-neurotoxic plasminogen activators are administered to a mammal from at least 3 hours after onset of a stroke. Another embodiment is directed to administration of non-neurotoxic plasminogen activators from at least 6 hours after onset of a stroke. In one embodiment of the invention, non-neurotoxic plasminogen activators are administered from at least 9 hours after onset of a stroke. Another embodiment of the invention contemplates the first administration of non-neurotoxic plasminogen activators to a mammal during a period between 3 and 6 hr after onset of a stroke. Yet another embodiment of the invention contemplates the first administration to mammal of non-neurotoxic plasminogen activators during a period between 6 and 9 hr after onset of a stroke.

DSPA as well as further non-neurotoxic plasminogen activators do not show tissue damaging side effects. However, one embodiment of the invention contemplates the administration of non-neurotoxic plasminogen activators in combination with a neuroprotective agent for the treatment of stroke in order to limit the tissue damages induced by the glutamate occurring naturally in the human body. Treatment of stroke as used in this application can mean reducing the effects associated with stroke, and/or reducing its severity. An example of successful treatment includes increased blood flow to the affected area compared to blood flow in the absence of administration of non-neurotoxic plasminogen activators. Neuroprotective agents inhibiting the glutamate receptor competitively or non-competitively can be used. Useful combinations include known inhibitors of the glutamate receptors, e.g., the NMDA type, the kainic acid type or the quisqualate type, as for example APV, APH, phencyclidine, MK-801, dextrorphane or cetamine.

Further, a combination with cations can also be used, since cations, for example Zn-ions, block the cation channel regulated by the glutamate receptor and can therefore reduce neurotoxic effects.

In another embodiment, non-neurotoxic plasminogen activators can be combined with at least one further therapeutic agent or with a pharmaceutically tolerable carrier. The combination with a therapeutic agent that supports the reduction of tissue damage by vitalizing the cells can also be used, since it contributes to the regeneration of already damaged tissue or serves for the prevention of further stroke incidents. Examples include combinations with antibiotics as quinones, anticoagulants as heparin or hirudin as well as with citicholine or acetylsalicylic acid.

A combination with at least one thrombin inhibitor can also be used. For example, thrombomodulin and thrombomodulin analogs like for example solulin, triabin or pallidipin can be used. Further, combinations with anti-inflammatory substances can also be employed in the invention since they influence the infiltration by leucocytes.

Various embodiments of the invention can be summarized as follows:

A method is provided for treating stroke in a mammal, comprising administering to the mammal a therapeutically effective amount of a plasminogen activating factor (PAF), wherein the PAF's activity is enhanced by more than 650 fold in the presence of fibrin.

According to an embodiment of the invention, the plasminogen activating factor comprises at least a part of a cymogene triade, wherein the part of the cymogene triade comprises at least a histidine residue or serine residue which interacts with an aspartate residue.

According to a further embodiment of the invention the serine residue, if present, is located at a position at least partially homologous to 292, the histidine residue, if present, is located at a position at least partially homologous to 305, and the aspartate residue is located at a position at least partially homologous to 447 of t-PA.

According to a further embodiment of the invention the plasminogen activating factor is t-PA mutant t-PA/R275E; t-PA/R275E, F305H; or t-PA/R275E, F305H, A292S.

According to a further embodiment of the invention the plasminogen factor has a point mutation at Asp194, or at an aspartate at a position homologous to Asp194, wherein the point mutation reduces catalytically active conformation stability of the plasminogen activating factor in the absence of fibrin.

According to a further embodiment of the invention Asp194 is substituted by glutamate or asparagine.

According to a further embodiment of the invention the plasminogen activating factor comprises at least one mutation in its autolysis loop, wherein the mutation reduces the functional interactions between plasminogen and plasminogen activating factor in the absence of fibrin.

According to a further embodiment of the invention at least one mutation in the autolysis loop affects the amino acid positions 420 to 423 of wild type t-PA or homologous positions.

According to a further embodiment of the invention at least one mutation is L420A, L420E, S421G, S421E, P422A, P422G, P422E, F423A or F423E.

According to a further embodiment of the invention the plasminogen activating factor is a cymogene comprising at least one point mutation preventing the catalysis by plasmin.

According to a further embodiment of the invention at least one point mutation is located at position 15 or 275 of t-PA, or at a homologous position.

According to a further embodiment of the invention wherein glutamate is at position 15 or 275.

According to a further embodiment of the invention the plasminogen activating factor is vampire bat saliva plasminogen activating factor (DSPA).

According to a further embodiment of the invention the PAF is administered from at least 3 hours after onset of a stroke.

According to a further embodiment of the invention the PAF is administered from at least 6 hours after onset of a stroke.

According to a further embodiment of the invention the PAF is administered from at least 9 hours after onset of a stroke.

According to a further embodiment of the invention stroke onset in the patient is not exactly determined.

In a further embodiment of the invention a tissue plasminogen activating factor (PAF) is provided with an autolysis loop comprising His420, Asn421, Ala422 and Cys423.

According to a further embodiment of the invention the PAF further comprises a point mutation at position 194, which reduces catalytically active conformation stability of the PAF when fibrin is absent.

According to a further embodiment of the invention the position 194 comprises Phe194.

According to a further embodiment of the invention the PAF comprises at least one point mutation that prevents plasmin catalysis.

According to a further embodiment of the invention the point mutation is Glu275.

According to a further embodiment of the invention a modified urokinase is provided having enzymatic activity in presence of fibrin enhanced by more than 500-fold compared to wild type urkinase.

According to a further embodiment of the invention a urokinase is provided comprising an autolysis loop comprising Va1420, Thr421, Asp422 and Ser423. This urokinase might comprise a point mutation at position 194, wherein the point mutation reduces the stability of the catalytic active conformation of the urokinase in absence of fibrin.

The point mutation can be Glu194 and can further comprise at least one point mutation that prevents plasmin catalysis. This point mutation can be Ile275.

The urokinase can consist of the amino acid sequence of SEQ ID NO:2.

In a further embodiment the invention provides a pharmaceutical composition comprising this urokinase.

Below the mode of application and formulation according to the invention is outlined by means of distinct therapeutic examples.

Other than in the operating examples, or where otherwise indicated, all numbers expressing quantities used in the specification and claims are to be understood as being modified in all instances by the term “about.” Accordingly, unless indicated to the contrary, the numerical parameters set forth in the present specification and attached claims are approximations that may vary depending upon the desired properties sought to be obtained by the present invention. It should be understood that the exact numerical values disclosed also form embodiments of the invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### EXAMPLES

### Comparative Examination of t-PA and DSPA

A. Methods

1. Animals

Wild-type mice (c57/Black 6) and t-PA deficient mice (t-PA−/−mice) (c57/Black 6) (Carmeliet et al., 1994) were supplied by Dr. Peter Carmeliet, Leuven, Belgium.

2. Protein Extraction from Brain Tissue

The assessment of proteolytic activity in brain tissue following infusion of either t-PA or DSPAα1 was performed by zymographic analysis (Granelli-Piperno and Reich, 1974). After an infusion over a period of seven days into the hippocampus, mice were anaesthetised, then transcardially perfused with PBS and the brains removed. The hippocampus region was removed, transferred to eppendorf tubes and incubated in an equal volume (w/v) (approx. 30-50 μm) of 0.5% NP-40 lysis buffer containing no protease inhibitors (0.5% NP-40, 10 mM Tris-HCl pH 7.4, 10 mM NaCL, 3 mM MgCl₂, 1 mM EDTA). The brain extracts were homogenized by means of a hand-held glass homogeniser and left on ice for 30 minutes. The samples were then centrifuged and the supernatant was removed. The amount of proteins present was determined (Bio-Rad-reagent).

3. Zymographic Analysis of the Proteases

The proteolytic activity in the samples and the brain tissue extracts was determined by zymographic analysis according to the method of Granelli, Piperno and Reich (1974). The samples with recombinant proteins (up to 100 nM) or the brain tissue extracts (20 μg) were subjected to a (10%) SDS-PAGE under non-reducing conditions. The gels were removed from the plates, washed in 1% triton X 100 for 2 hours and then overlaid onto an agarose gel containing polymerized fibrinogen and plasminogen (Granelli, Piperno and Reich, 1974). The gels were incubated at 37° C. in a humified chamber until proteolysed zones appeared.

4. Intra-Hippocampal Infusion of t-PA, DSPA and Subsequent Injection of Kainic Acid

The kainic acid injury model was based on studies of Tsirka et al. (1995). The animals were injected intraperitoneally (i. p.) with atropine (4 mg/kg) and then anaesthetised with an i. p. injection of sodium pentobarbitol (70 mg/kg). Afterwards mice were placed in a stereotaxic frame and a micro-osmotic pump (Alzet model 1007D, Alzet Calif. USA) containing 100 μl of either PBS or recombinant human t-PA (0.12 mg/ml, 1.85 μM) or DSPAα1 (1.85 μM) was implanted subcutaneously between the shoulder blades. The pumps were connected via sterile tubes to a brain cannula and inserted through a burr opening made through the skull at coordinates bregma −2.5 mm, midiolateral 0.5 mm and dorsoventral 1.6 mm in order to introduce the liquid near the midline. The cannula was fixed at the desired position and the pumps were allowed to infuse the respective solutions at a rate of 0.5 μl per hour for a total of 7 days.

Two days after infusion of the proteases the mice were reanaesthetised and again placed in the stereotaxic frame. Afterwards 1.5 nmol of kainic acid (KA) in 0.3 μl PBS was injected unilaterally into the hippocampus. The coordinates were: bregma-2.5 mm, medial-lateral 1.7 mm and dorsoventral 1.6 mm. The excitotoxin (KA) was delivered for a duration of 30 seconds. After the kainic acid treatment the injection needle remained at these coordinates for further 2 minutes in order to prevent a reflux of the liquid.

5. Brain Processing Procedure

Five days after KA injection, the animals were anaesthetised and transcardially perfused with 30 ml PBS followed by 70 ml of a 4% paraformaldehyd solution, post fixed in the same fixative followed by incubation in 30% sucrose for further 24 hours. Coronal sections (40 μm) of the brain were then cut on a freezing microtome and either counter-stained with thionin (BDH, Australia) or processed for immunohistochemical examination as described below.

6. Quantification of Neuronal Loss within the Hippocampus

The quantification of neuronal loss in the CA1-CA3 hippocampal subfields was performed as previously described (Tsirka et al., 1995; Tsirka et al., 1996). Five consecutive parts of the dorsal hippocampus from all treatment groups were prepared taking care that the parts indeed comprised the place of the CA-injection and lesion area. The hippocampal subfields (CA1-CA3) of these sections were traced by means of camera lucida drawings of the hippocampus. The entire lengths of the subfields was measured by comparison to 1 mm standards traced under the same magnification. The lengths of tissue with viable pyramidal neurons (having normal morphology) and lengths of tissue devoid of neurons (no cells present, no thionin staining) was determined. The lengths, representing intact neurons and neuronal losses over each hippocampal subfield were averaged across sections and the standard deviations were determined.

7. Intra-Striatal NMDA Excitotoxic Lesions with or Without t-PA or DSPA

Wild type mice (c57/Black 6) were anaesthetised and placed in a sterertaxic frame (see above). Mice then received an unilateral injection of 50 nmol NMDA in the left stratum, injected alone or in combination with either 46 μM rt-PA or 46 μM DSPAα1. As controls t-PA and DSPA were also injected alone (both at a concentration of 46 μM). The injection coordinates were: bregma −0.4 mm, midiolateral 2.0 mm and dorsoventral 2.5 mm. The solutions (1 μl total volume for all treatments) were transferred over a period of 5 minutes at a rate of 0.2 μl/min and the needle was left in place for further 2 minutes after the injection in order to minimize the reflux of fluid. After 24 hours the mice were anaesthetised and perfused transcardially with 30 ml PBS followed by 70 ml of a 4% paraformaldehyd solution, post fixed in the same fixative for 24 hours with followed by incubation in 30% sucrose for further 24 hours. Brains were then cut (40 μm) on a freezing microtome and mounted onto gelatin coated glass slides.

8. Quantification of the Lesion Volume Following NMDA Injection

The quantification of the striatal lesion volume was performed using the method described by Callaway et al. (2000). Ten consecutive coronal sections spanning the lesioned area were prepared. The lesioned area was visualised using the Callaway method and the lesion volume was quantified by the use of a micro computer imaging device (MCID, Imaging Research Inc., Brock University, Ontario, Canada).

9. Immunohistochemistry

Immunohistochemistry was performed using standard methodologies. Coronal sections were immersed in a solution of 3% H₂O₂ and 10% methanol for 5 minutes followed by an incubation in 5% normal goat serum for 60 minutes. The sections were incubated over night either with an anti-GFAP antibody (1:1.000; Dako, Carpinteria, Calif., USA) for the detection of astrocytes, with an anti-MAC-1 antibody (1:1.000; Serotec, Raleigh, N.C., USA) for the detection of microglia or with polyclonal anti-DSPA antibodies (Schering A G, Berlin). After rinsing, the sections were incubated with the appropriate biotinylated secondary antibodies (Vector Laboratories, Burlingame, Calif., USA). This was followed by a final incubation with avidin/biotin-complex (Vector Laboratories, Burlingame, Calif., USA) for 60 minutes before visualisation with 3,3′-diaminebebcidine/0.03% H₂O₂. Sections were then mounted on gelatin coated slides, dried, dehydrated and coverslipped with permount.

10. Enhancement of the Tissue Lesions Induced by NMDA Injection by Intravenous Administration of t-PA or DSPA

In order to induce tissue lesions within the striatum, mice were stereotactically injected with NMDA. Six hours after NDMA injection t-PA or DSPA (100 μl; 10 mg/kg) were injected via the tail vein. As control 100 μl 0.9% NaCl were injected and subsequently PBS infused. After 28 hours the animals were killed and the lesion volume was determined.

In a second trial sets of animals including up to 15 mice were injected accordingly, with an infusions of t-PA or DSPA 24 hours after NMDA injection and a subsequent determination of the tissue damage. As proof of the presence of DSPA within the brain, coronal sections were stained with an anti-DSPA antibody according to standard methods.

B. Results

1. Infusion of t-PA or DSPA Disperses into the Hippocampus of t-PA −/− Mice and Retains Proteolytic Activity

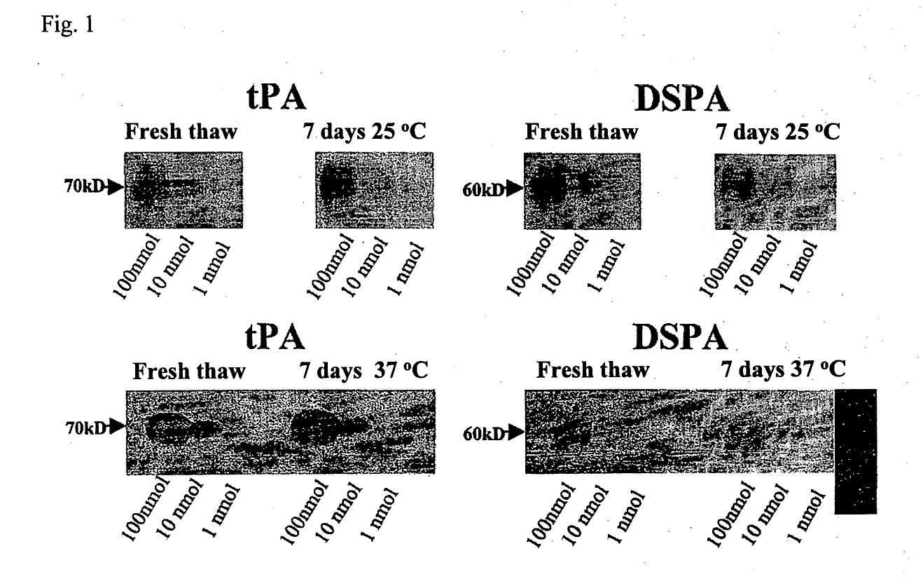

The initial experiments were designed to confirm that both DSPA and t-PA retain their proteolytic activity for the 7 day period of the infusion. To this end, aliquots of t-PA and DSPA (100 nmol) were incubated at 37° C. and at 30° C. for 7 days in a water bath. In order to determine the proteolytic activity, 5 fold serial dilutions of the probes were subjected to SPS-PAGE under non-reducing conditions and proteolytic activity was assessed by zymographic analyses. An aliquot of t-PA and DSPA which had been kept frozen for a period of 7 days was used as a control. As can be seen in FIG. 1 there was only a minor loss of DSPA or t-PA activity at an incubation with either 25° C. or 37° C. over this period of time.

2. t-PA and DSPA Activity is Recovered in Hippocampal Extracts Prepared from t-PA −/− Mice Following Infusion

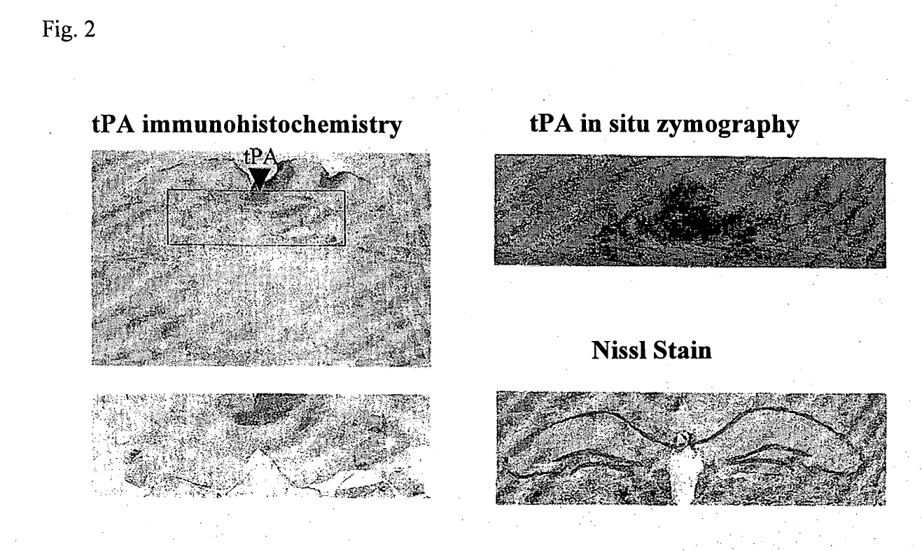

First it had to be confirmed that the infused proteases were present in the brain of the infused animals and also retained their proteolytic activity while being in this compartment. To address this point, t-PA −/− were infused for seven days with either t-PA or DSPA (see above). Mice were then transcardially perfused with PBS and the brains removed. The ipsilateral and contralateral hippocampal regions were isolated as well as a region of the cerebellum (taken as a negative control). Tissue samples (20 μg) were subjected to SDS-PAGE and zymographic analysis according to the description in the methods section. As can be seen in FIG. 2, both t-PA and DSPA activities were detected in the ipsilateral region of the hippocampus, while some activity was also detected on the contralateral side. This indicates that the infused proteases not only retained their activity in the brain but had also diffused within the hippocampal region. As a control, no activity could be detected in the extract prepared from the cerebellum.

3. Immunohistochemical Assessment of DSPA

To further confirm that DSPA had indeed diffused into the hippocampal region, coronal brain sections of t-PA −/− mice were analysed immunohistochemically after DSPA infusion. DSPA-antigen was detected in the hippocampal region with the most prominent staining in the area of the infusion site. This result confirms that the infused DSPA is soluble and is indeed present in the hippocampus.

4. DSPA Infusion does not Restore Kainic-Acid Mediated Neurodegeneration In Vivo

t-PA −/− mice are characteristically resistant to kainic acid (KA) mediated neurodegeneration. However, intrahippocampal infusion of rt-PA completely restores the sensitivity to KA-mediated injury. To determine whether DSPA could be substituted for t-PA in this model, t-PA −/− mice were infused intrahipocampically with either t-PA or DSPA using a mini-osmotic pump. For both groups 12 mice were tested. 2 days later the animals were injected with kainic acid and left to recover. 5 days later the animals were killed and the brains removed and prepared (see above). As controls, t-PA −/− mice were also infused with PBS prior to KA treatment (N=3).

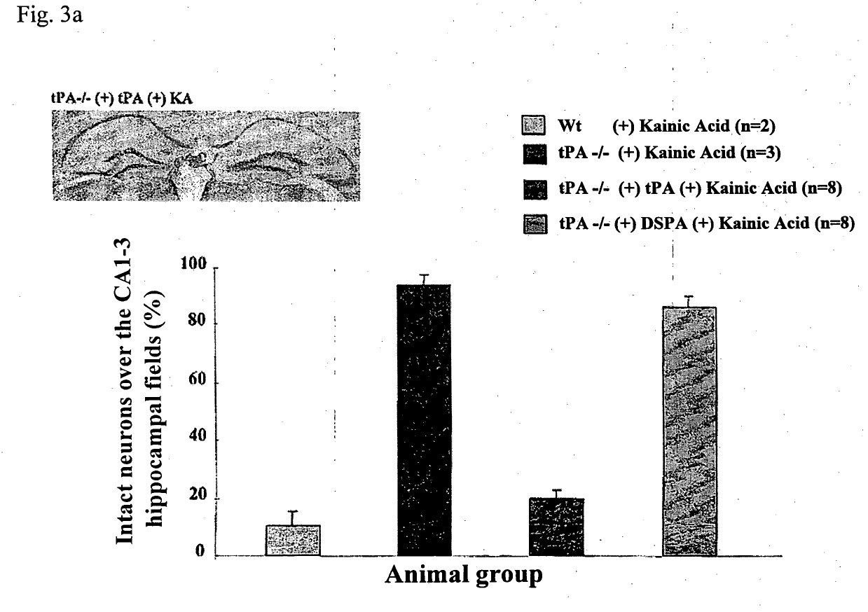

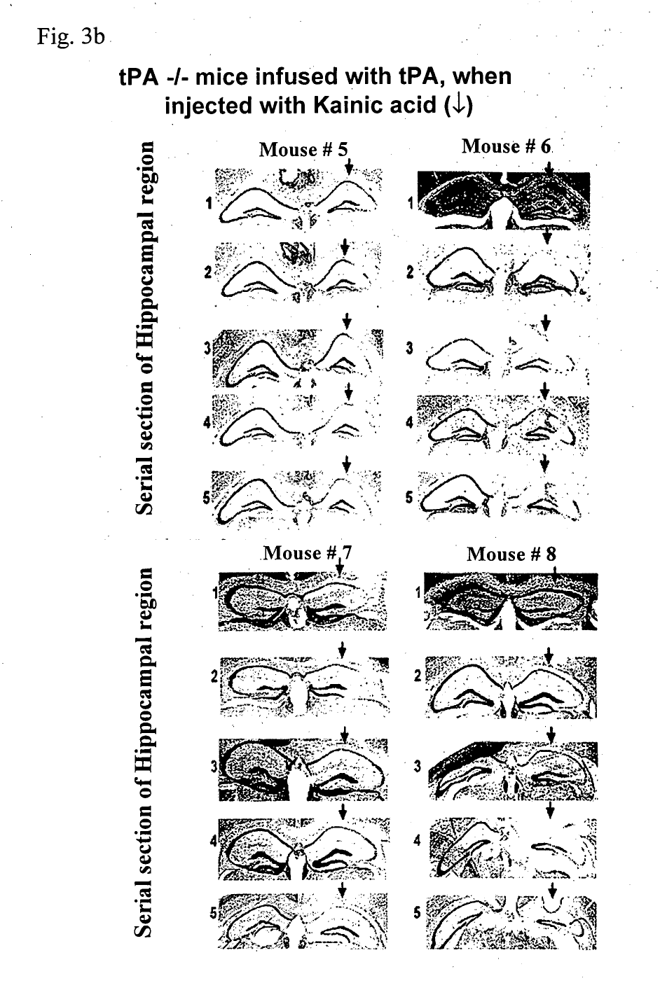

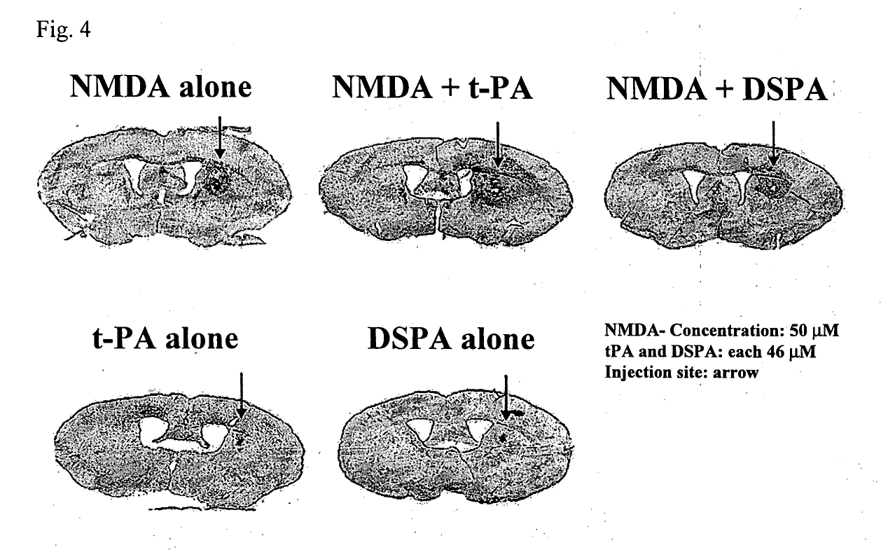

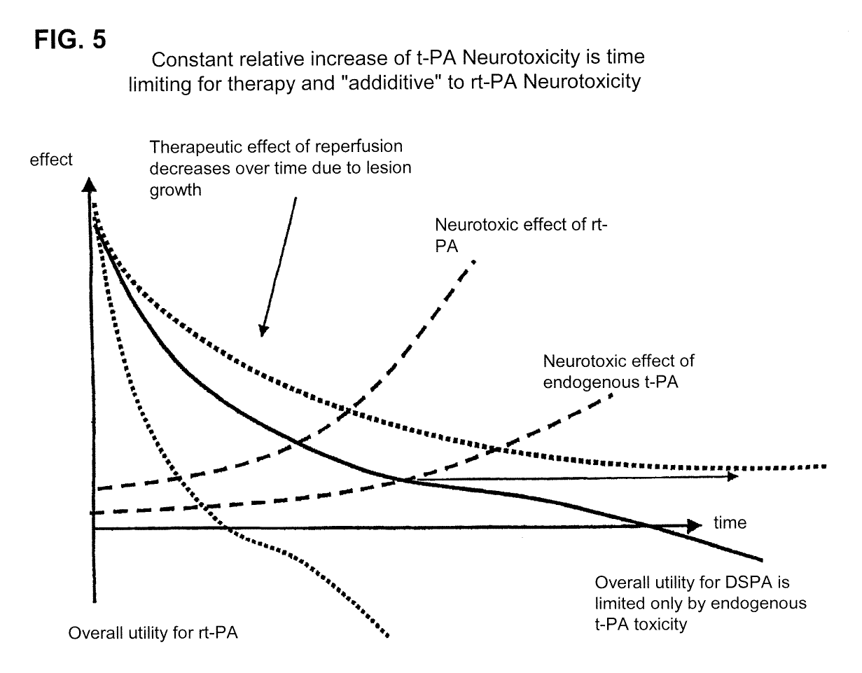

Coronal brain sections were prepared and the neurons detected by Nissl staining. As shown in FIGS. 3 and 4, t-PA −/− mice infused with PBS were resistant to subsequent challenge with KA. However, infusion of recombinant t-PA restored sensitivity to KA treatment. In contrast, infusion of the same concentration of DSPA into the hippocampal region did not alter the sensitivity of the animals to KA.

A quantitation of those results was based on data obtained from 12 mice in each group. In 2 of the 12 mice infused with DSPA a small extend of neurodegeneration was observed. The reason for that in unclear and possibly not related to the presence of DSPA. The combined data consider this minor effect that was observed in the case of these 2 animals. All 12 mice treated with t-PA were sensitive against the KA treatment. These results show that in case of an infusion of t-PA or DSPAα1 in equimolar concentrations only the administering of t-PA led to the restoration of sensitivity to KA induced neurodegeneration.

5. DSPA Infusion does not Result in Microglial Activation

The restauration of the KA sensitivity of the t-PA −/− mice caused by a t-PA infusion also results in a microglia activation (Rogove et al., 1999). To assess the degree of microglial activation following t-PA or DSPA infusion and subsequent KA treatment, coronal sections of mice were subjected to an immunohistochemical staining for activated microglia cells using the Mac-1 antibody. The restauration of KA sensitivity following t-PA infusion resulted in a clear increase in Mac-1 positive cells. This was not observed in mice infused with DSPA. Hence, the presence of DSPA does not result in the activation of microglia cells following KA treatment.

6. Titration of DSPA and t-PA in the Mice Hippocampus Region.

The concentration of t-PA used for the infusion was based on the concentration described by Tsirka et al. (1995) (100 μl of 0.12 mg/ml [1.85 μM]). The KA-injury experiments were repeated using a 10-fold lower of t-PA (0.185 μM) and a 10-fold higher amount of DSPA (18.5 μM). The lower t-PA concentration was still able to restore the sensitivity to KA treatment (n=3). Of special interest was the finding that the infusion of 10 fold increased DSPA concentration only caused a little neuronal loss following KA treatment. These data strongly point out that DSPA does not lead to an increase of sensitivity to KA.

7. Effect of t-PA and DSPA on NMDA-Dependent Neurodegeneration in Wild Type Mice

The effects of t-PA and DSPA were also examined in a model of neurodegeneration in wild type mice. The injection of t-PA in the striatum of these mice probably led to an increase of the neurodegenerative effects caused by the glutamate analogue NMDA (Nicole et al., 2001).

NMDA was injected into the striatal region of wild type mice in the presence of t-PA or DSPA (each 46 μM) with a total volume of 1 μl. After 24 hours the brains were removed and the size of the lesions was quantified according to the Callaway method (Callaway et al., 2000) (see above). As can be seen in FIG. 4, injection of NMDA alone caused a reproducible lesion in all treated mice (N=4). When t-PA and NMDA were applied together, the size of the lesions was increased about 50% (P<0.01, n=4). In a clear contrast the co-injection of NMDA and the same concentration of DSPA did not lead to an increase in lesion size compared to NMDA alone.

Injection of t-PA or DSPA alone did not lead to a detectable neurodegeneration. The lacking effect of t-PA when being administered alone is consistent with the results of Nicole et al. (2001). These data show that the presence of DSPA does not increase neurodegeneration even during a neurodegenerative event.

In order to confirm that the injection of DSPA had indeed spread into the hippocampal region, immunohistochemistry was performed on coronal sections by use of the DSPA antibody. The examination showed that DSPA did indeed enter the striatal region.

Kinetic Analysis of the Plasminogen Activation by Indirect Chromogen Test

Indirect chromogen tests of the t-PA activity were performed using the substrate Lys-plasminogen (American Diagnostica) and spectrocyme PL (American Diagnostica) according to Madisan E. L., Goldsmith E. J., Gerard R. D., Gething M.-J., Sambrook J. F. (1989) Nature 339 721-724; Madison E. L O., Goldsmith E. J., Gething M. J., Sambrook J. F. and Bassel-Duby R. S. (1990) Proc. Natl. Acad. Sci. U.S:A 87, 3530-3533 as well as Madison E. L., Goldsmith E. J., Gething M. J., Sambrook J. F. and Gerard R. D. (1990) J. Biol. Chem. 265, 21423-21426. Tests were performed both in the presence and absence of the co-factor DESAFIB (American Diagnostica). DESAFIB is a preparation of soluble fibrin monomeres gained by the cleavage of highly pure human fibrinogen with the protease batroxobin. Batroxobin cleaves the Arg¹⁶-Gly¹⁷-binding in the Aα-chain of fibrinogen and thereby releases fibrinopeptid A. The resulting des-AA-fibrinogen representing fibrin I monomers is soluble in the absence of the peptide Gly-Pro-Arg-Pro (SEQ ID NO:3). The concentration of Lys-plasminogen was varied from 0.0125 up to 0.2 μM in the presence of DESAFIB and from 0.9 to 16 μM in absence of the co-factor.

Indirect Chromogen Tests in the Presence of Different Stimuli.

Indirect chromogen standard tests were performed according to the publications cited above. Probes of 100 μl total volume containing 0.25-1 ng enzyme, 0.2 μM Lys-plasminogen and 0.62 mM spectrocyme PL were used. The tests were performed either in the presence of buffer, 25 μg/ml DESAFIB, 100 μg/ml cyanogen bromide fragments of fibrinogen (American Diagnostica) or 100 μg/ml of the stimulatory 13 amino acid peptide P368. The analysis were performed in microtiter-plates and the optic density was determined at a wave length of 405 nm every 30 seconds for 1 hour in a “Molecular Devices Thermomax”. The reaction temperature was 37° C.

8. DSPA also in case of Intravenous Application does not Cause an Increase of Neural Tissue Damages

In the striatum of mice, tissue lesions were induced by the injection of NMDA, followed by the intravenous application of t-PA or DSPA 6 or 24 hours thereafter. In comparison to a negative control, the experimental animals showed an increase of about 30% of the damaged tissue area produced by the NMDA-injection, when t-PA was given as an intravenous infusion 24 hours after the NMDA-injection; this was in contrast to DSPA, which did not cause such an increase of the tissue damages (see FIG. 14). By the staining of coronal sections with an anti-DSPA-antibody, it was possible to detect, that the DSPA intravenously administered 24 hours after the NMDA-injection had permeated into the damaged tissue areas (see FIG. 15). In case of a corresponding intravenous application of t-PA or DSPA 6 hours after the NMDA-injection, an increase of the damaged tissue area was not yet to be detected. This may have its reason in that the blood-brain barrier at this moment of the t-PA or DSPA application still provided a sufficient barrier function. DSPA thus also in case of intravenous application does not display neurotoxic side effects.

9. Dose Escalation of Desmoteplase for Acute Ischemic Stroke Study (DEDAS)

List of Tables: Table 6: Characteristics of patients
Table 7: Safety (ITT population)
Table 8: Efficacy
Table 9: Characteristics of patients in the 6-9 h time window
Table 10: Efficacy of patients in the 6-9 h time window

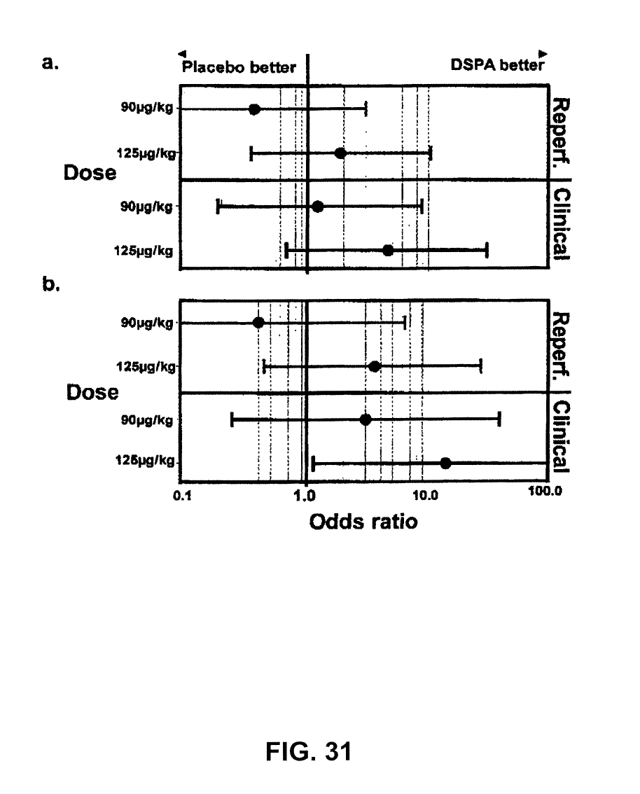

List of Figures: FIG. 31: Reperfusion and Good Clinical Outcome Univariate Odds Ratios over Placebo

Figure Legend

FIG. 31: Univariate odds ratios over placebo regarding reperfusion (gray bars) and good clinical outcome

Intravenous (IV) thrombolytic treatment of acute ischemic stroke is currently limited to recombinant tissue plasminogen activator (rtPA) administered within 3 hours after symptom onset.¹ The references cited in superscripts can be found in the References section of this application preceded by the corresponding number in parenthesis. All rtPA trials employed routine brain CT for patient selection.

Newer imaging technologies suggest that salvageable brain may be present for a number of hours in many patients with acute ischemic stroke.⁶⁻⁹ The Desmoteplase in Acute Stroke Study (DIAS)¹³ selected patients for IV thrombolysis up to 9 hours from stroke onset based on a perfusion/diffusion mismatch on MRI. DIAS reported a low rate of sICH and evidence of reperfusion and clinical efficacy using desmoteplase and MRI. The Dose Escalation of Desmoteplase for Acute Ischemic Stroke Study (DEDAS) evaluated the safety and efficacy of two doses of desmoteplase (DSPAα1) that were associated with a favorable benefit-risk profile in the DIAS trial.

Patients and Methods

Methods and procedures of DEDAS were largely identical to DIAS.¹³ The specifics of DEDAS and a brief overview are reported here. Patients and desmoteplase dosages: Twenty-five centers (21 U.S, and 4 Germany) participated in DEDAS between March 2003 and October 2004. The protocol and all amendments received IRB approval at each center, and written informed consent was obtained from all patients or their legal representatives.

DEDAS was a randomized, placebo-controlled, bodyweight-adjusted dose-escalation study starting at a dose of 90 μg/kg Desmoteplase (i.e. DSPAα1). One additional dose, 125 μg/kg, was evaluated. Each dose tier included 15 desmoteplase patients and four placebo patients. No stratification was implemented. Patients were randomized to desmoteplase or placebo via an interactive voice response system. Study drug was administered as an IV bolus over 1-2 minutes.

Main Inclusion Criteria: Age 18 through 85 years, stroke onset within 3 through 9 hours; baseline NIHSS scores 4 to 20; at least 20% perfusion/diffusion mismatch (as evaluated by visual inspection) with perfusion deficit >2 cm in diameter and involving the cerebral cortex on baseline MRI. Randomization was based on investigator readings of MRI. Analyses were based on blinded central imaging readings (Perceptive Informatics, Inc.).

Exclusion criteria were similar to those adopted by other thrombolytic trials including any hemorrhage on baseline CT. Isolated internal carotid artery (ICA) occlusions were excluded.

Imaging examinations: MRI was performed at screening, 4-8 hours post-treatment and 30 days follow-up. After 24 hours, a computed tomography (CT) was performed on all patients to document any intracranial bleeding.

Safety endpoints: The primary safety endpoint was the rate of sICH defined as any ICH associated with a worsening of four points or more on the NIHSS and confirmed by CT within 72 hours of treatment. Other safety outcomes included mortality, anaphylaxis and major systemic bleeding-defined as bleeding considered to be life-threatening-which is the requirement to administer 2 or more units of packed red blood cells or a hemoglobin (Hb)-drop by 40 g/L or more. Other adverse events and serious adverse events were also monitored.

Efficacy endpoints. The co-primary efficacy endpoints were reperfusion at 4-8 hours and clinical outcome at 90 days. Reperfusion was defined as either ≧30% reduction of mean transit time (MTT) volume of abnormality or ≧2 points improvement on the adapted magnetic resonance angiography (MRA) Thrombolysis in Myocardial Infarction (TIMI) scale. Good clinical outcome was defined based on a composite endpoint of ≧8 points improvement or scoring 0-1 on the NIHSS, 0-2 on the modified Rankin Scale (mRS), and a Barthel Index (BI) score of 75-100 at 90 days.

The primary analysis was the intention-to-treat (ITT). Secondary analyses were performed on a target population (TP) that included only those patients with MRI mismatch and no isolated ICA occlusion as determined by the central laboratory.

Concomitant medication: In the first 24 hours after administration of study drug, anticoagulants and antiplatelet agents were not allowed. The use of other thrombolytics was prohibited in the first 72 hours.

Results

Thirty-eight patients were randomized. One patient, randomized to 90 μg/kg desmoteplase, received no study drug and was excluded from all analyses. Hence, the ITT population included 37 patients (placebo, n=8; 90 μg/kg desmoteplase, n=14; 125 μg/kg demosteplase, n=15).

The desmoteplase and placebo groups were balanced with regard to age but not for time from symptom onset (longer time from onset in the 90 μg/kg dose group), DWI (diffusion weighted imaging) lesion volumes (larger in placebo), and baseline NIHSS (highest in placebo; Table 6). Four patients terminated the study prematurely, three due to death, one withdrew consent.

Twelve patients failed to meet the study defined MRI inclusion criteria as determined by the central laboratory. Six patients had an isolated ICA occlusion and another six had either no mismatch or no perfusion deficit. The majority of patients not meeting MRI criteria occurred in the 90 μg/kg dose tier (n=6), four occurred in the 125 μg/kg group and two in the placebo group. The TP sample size therefore was: n=6 placebo, n=8 90 μg/kg, and n=11 125 μg/kg (total TP N=25).

Safety (ITT)

Primary safety endpoint: No symptomatic ICHs were observed.

Other safety endpoints: Three deaths occurred within 90 days, one from each group. The placebo patient died 5 days after study drug administration secondary to stroke progression. The patient in the 90 μg/kg group died 53 days after study drug administration secondary to aspiration pneumonia, and the 125 μg/kg patient died at day 15 after study drug administration secondary to stroke progression.

Asymptomatic ICH (aICH)-based on 24-hour CT in all patients-occurred in 12.5% of placebo-treated patients and 35.7% and 40.0% of patients treated with 90 μg/kg or 125 μg/kg, respectively. Seventy-five percent of the aICHs occurred within 24 h after study drug administration. Major systemic hemorrhage occurred in 12.5% of patients receiving placebo and 14.3% and 13.3% of patients from the 90 μg/kg or 125 μg/kg desmoteplase groups, respectively. No anaphylactic reactions occurred (Table 7).

Efficacy

Reperfusion (ITT): MR-images of three patients treated with 90 μg/kg desmoteplase were either missing or not assessable to accurately judge reperfusion. Early reperfusion at 4-8 hours after treatment was observed in 37.5% of the placebo-treated patients (n=8) and in 18.2% and 53.3% of patients in the 90 μg/kg (n=11) and 125 μg/kg (n=15) groups, respectively (Table 8).

Primary Clinical Endpoint (ITT):

The rate of good outcome at 90 days was 25.0%, 28.2% and 60.0% of patients in the placebo (n=8), 90 μg/kg (n=14) and 125 μg/kg (n=15) desmoteplase groups, respectively (Table 8).

Target Population: Reperfusion

In the TP, the reperfusion rate increased for the 125 μg/kg group (n=11) compared to the ITT (63.6%) but decreased for placebo (33.3%; n=6) and for 90 μg/kg (16.7%; n=6)(see Table 8). Two patients in the 90 μg/kg group had MR-images not assessable for early reperfusion.

The reperfusion rates of the excluded patients were low: 1 out of 2 placebo patients, 1 out of 5 patients treated with 90 μg/kg (20.0%), and 1 out of 4 patients treated with 125 μg/kg (25.0%) demonstrated reperfusion, yielding an overall reperfusion rate of 22.2% in the desmoteplase patients with MRI exclusions.

Target Population: Primary Clinical Endpoint

The rate of good clinical outcome increased for the desmoteplase groups (37.5% and 72.7% for the 90 μg/kg [n=8] and 125 μg/kg [n=11] groups, respectively), but decreased for placebo (16.7%; n=6) when MRI violators were excluded. The clinical outcome in excluded patients was poor. Only 1 out of 2 placebo patients, 1 out of 6 patients treated with 90 μg/kg, and 1 out of 4 patients treated with 125 μg/kg had good clinical outcomes.

Additional Analyses of the data regarding the time window for therapeutic treatment are given in tables 9, 10 and 11.

### REFERENCES

(6) Baird A E, Benfield A, Schlaug G, Siewert B, Lovblad K O, Edelman R R, Warach S. Enlargement of human cerebral ischemic lesion volumes measured by diffusion-weighted magnetic resonance imaging. Ann Neurol. 1997; 41:581-589.
Baranes D, Lederfein D, Huang Y Y, Chen M, Bailey C H, Kandel E R. 1998. Tissue plasminogen activator contributes to the late phase of LTP and to synaptic growth in the hippocampal mossy fiber pathway. Neuron 21:813-25.
Bringmann P, Gruber D, Liese A, Toschi L, Kratzchmar J, Schleuning W D, Donner P. 1995. Structural features mediating fibrin selectivity of vampire bat plasminogen activators. J Biol Chem 270:25596-25603.
(9) Butcher K S, Parsons M, Macgregor L, Barber P A, Chalk J, Bladin C, Levi C, Kimber T, Schultz D, Fink J, Tress B, Donnan G, Davis S. Refining the perfusion-diffusion mismatch hypothesis. Stroke. 2005
Callaway J K, Knight M J, Watkins D J, Beart P M, Jarrott B, Delaney P M. 2000. A novel, rapid, computerized method for quantitation of neuronal damage in a rat model of stroke. J Neurosci Methods 102:53-60
Carmeliet P, Schoonjans L, Kieckens L, Ream B, Degen J, Bronson R, De Vos R, van den Oord J J, Collen D, Mulligan R C. 1994. Physiological consequences of loss of plasminogen activator gene function in mice. Nature 368:419-424.
Cartwright T. 1974. The plasminogen activator of vampire bat saliva. Blood 43:317-326.
Chalela J A, Kang D W, Luby M, Ezzeddine M, Latour L L, Todd J W, Dunn B, Warach S. Early magnetic resonance imaging findings in patients receiving tissue plasminogen activator predict outcome: Insights into the pathophysiology of acute stroke in the thrombolysis era. Ann Neurol. 2004; 55:105-112.
Chen Z L Strickland S. 1997. Neuronal death in the hippocampus is promoted by plasmin-catalysed degradation of laminin. Cell 91:917-925.
Chen, Z-L, Indyk, J. A., Bugge, T. H., Kombrinck, K. W., and Strickland S. 1999. Neuronal Death and blood-brain barrier breakdown after excitotoxic injury are independent processes. J. Neuroscience 19:9813-9820
Clark W M, Wissman S, Albers G W, Jhamandas J H, Madden K P, Hamilton S. Recombinant tissue-type plasminogen activator (Alteplase) for ischemic stroke 3 to 5 hours after symptom onset. The ATLANTIS study: A randomized controlled trial. Alteplase thrombolysis for acute noninterventional therapy in ischemic stroke. JAMA. 1999; 282:2019-2026.
(1) N Engl J. Med. 1995; Tissue plasminogen activator for acute ischemic stroke. The national institute of neurological disorders and stroke rt-pa stroke study group. 333:1581-1587.
Frey U, Muller M, Kuhl D. 1996. A different form of long-lasting potentiation revealed in tissue plasminogen activator mutant mice. J Neurosci 16:2057-2063.
Gardell S J, Duong L T, Diehl R E, York J D, Hare T R, Register R B, Jacobs J W, Dixon R A, Friedman P A. 1989. Isolation, characterization, and cDNA cloning of a vampire bat salivary plasminogen activator. J Biol Chem 264:17947-17952.
Gardell S J, Ramjit D R, Stabilito I I, Fujita T, Lynch J J, Cuca G C, Jain D, Wang S P, Tung J S, Mark G E, et al. 1991. Effective thrombolysis without marked plasminemia after bolus intravenous administration of vampire bat salivary plasminogen activator in rabbits. Circulation 84:244-253.
Granelli-Piperno, A and Reich, E. 1974. A study of proteases and protease inhibitor complexes in biological fluids. J. Exp. Med. 148: 223-234.
Hacke W, Kaste M, Fieschi C, Toni D, Lesaffre E, von Kummer R, Boysen G, Bluhmki E, Hoxter G, Mahagne M H, et al. Intravenous thrombolysis with recombinant tissue plasminogen activator for acute hemispheric stroke. The European cooperative acute stroke study (ECASS). JAMA. 1995; 274:1017-1025.
Hacke W, Kaste M, Fieschi C, von Kummer R, Davalos A, Meier D, Larrue V, Bluhmki E, Davis S, Donnan G, Schneider D, Diez-Tejedor E, Trouillas P. Randomised double-blind placebo-controlled trial of thrombolytic therapy with intravenous alteplase in acute ischaemic stroke (ECASS II). Second European-australasian acute stroke study investigators. Lancet. 1998; 352:1245-1251.
Hacke W, Donnan G, Fieschi C, Kaste M, von Kummer R, Broderick J P, Broft T, Frankel M, Grotta J C, Haley E C, Jr., Kwiatkowski T, Levine S R, Lewandowski C, Lu M, Lyden P, Marler J R, Patel S, Tilley B C, Albers G, Bluhmki E, Wilhelm M, Hamilton S. Association of outcome with early stroke treatment: Pooled analysis of ATLANTIS, ECASS, and NINDS rt-PA stroke trials. Lancet. 2004; 363:768-774.
(13) Hacke W, Albers G, Al-Rawi Y, Bogousslavsky J, Davalos A, Eliasziw M, Fischer M, Furlan A, Kaste M, Lees K R, Soehngen M, Warach S. The desmoteplase in acute ischemic stroke trial (DIAS): A phase II MRI-based 9-hour window acute stroke thrombolysis trial with intravenous desmoteplase. Stroke. 2005; 36:66-73.

Huang Y Y, Bach M E, Lipp H P, Zhuo M, Wolfer D P, Hawkins R D, Schoonjans L, Kandel E R, Godfraind J M, Mulligan R, Collen D, Carmeliet P. 1996. Mice lacking the gene encoding tissue-type plasminogen activator show a selective interference with late-phase long-term potentiation in both Schaffer collateral and mossy fiber pathways. Proc Natl Acad Sci USA. 93:8699-86704.
(7) Karonen J O, Vanninen R L, Liu Y, Ostergaard L, Kuikka J T, Nuutinen J, Vanninen E J, Partanen P L, Vainio P A, Korhonen K, Perkio J, Roivainen R, Sivenius J, Aronen H J. Combined diffusion and perfusion mri with correlation to single-photon emission ct in acute ischemic stroke. Ischemic penumbra predicts infarct growth. Stroke. 1999; 30:1583-1590.
Kratzschmar J, Haendler B, Langer G, Boidol W, Bringmann P, Alagon A, Donner P, Schleuning W D. 1991. The plasminogen activator family from the salivary gland of the vampire bat Desmodus rotundus: cloning and expression. Gene 105:229-237.
Liberatore G T, Samson A, Bladin C, Schleuning W D, Medcalf R L. Vampire bat salivary plasminogen activator (desmoteplase): A unique fibrinolytic enzyme that does not promote neurodegeneration. Stroke. 2003; 34:537-543.

Liu D, Cheng T, Guo H, Fernandez J A, Griffin J H, Song X, Zlokovic B V. Tissue plasminogen activator neurovascular toxicity is controlled by activated protein C. Nat. Med. 2004; 10:1379-1383.
Madani R, Hulo S, Toni N, Madani H, Steimer T, Muller D, Vassalli J D. (1999) Enhanced hippocampal long-term potentiation and learning by increased neuronal expression of tissue-type plasminogen activator in transgenic mice. EMBO J. 18:3007-3012
Melloft M J, Stabilito I I, Holahan M A, Cuca G C, Wang S, Li P, Barrett J S, Lynch J J, Gardell S J. 1992. Vampire bat salivary plasminogen activator promotes rapid and sustained reperfusion concomitant systemic plasminogen activation in a canine model of arterial thrombosis. Arterioscler. Thromb. 12:212-221.
Molina C A, Alvarez-Sabin J, Montaner J, Abilleira S, Arenillas J F, Coscojuela P, Romero F, Codina A. Thrombolysis-related hemorrhagic infarction: A marker of early reperfusion, reduced infarct size, and improved outcome in patients with proximal middle cerebral artery occlusion. Stroke. 2002; 33:1551-1556.
Muschick, P., Zeggert D., Donner, P., Witt, W. 1993. Thrombolytic properties of Desmodus (vampire bat) plasminogen activator DSPAα1, Alteplase and streptokinase following intravenous bolus injection in a rabbit model of carotid artery thrombolysis. Fibrinolysis 7: 284-290.

Müller, C. M., Griesinger, C. B. 1998. Tissue plasminogen activator mediates reverse occlusion plasticity in visual cortex. Nature Neuroscience. 1: 47-53.
National Institute of Neurological Disorders and stroke rt-PA study group. 1995. New. Engl. J. Med. 333: 1581-1587
Nicole, O., Docagne, F., Ali, C., Margaill, I., Carmeliet, P., MacKenzie, E. T., Vivien, D. & Buisson, A. (2001) The proteolytic activity of tissue-plasminogen activator enhances NMDA receptor-mediated signaling. Nat Med 7, 59-64.
Reddrop C, Moldrich R X, Beart P M, Farso M, Liberatore G T, Howells D W, Petersen K U, Schleuning W D, Medcalf R L. Vampire bat salivary plasminogen activator (desmoteplase) inhibits tissue-type plasminogen activator-induced potentiation of excitotoxic injury. Stroke. 2005; 36:1241-1246.

Rogove A D, Siao C, Keyt B, Strickland S, Tsirka S E. 1999. Activation of microglia reveals a non-proteolytic cytokine function for tissue plasminogen activator in the central nervous system. J Cell Sci 112:4007-4016.
Schleuning W D, Alagon A, Boidol W, Bringmann P, Petri T, Kratzschmar J, Haendler B, Langer G, Baldus B, Wift W, et al. 1992. Plasminogen activators from the saliva of Desmodus rotundus (common vampire bat): unique fibrin specificity. Ann N Y Acad Sci 667:395-403.
Seeds, N. W., Williams, B. L., Bickford, P. C. 1995. Tissue plasminogen activator induction in purkinje neurons after cerebellar motor learning. Science. 270:1992-1994.
Stewart R J, Fredenburgh J C, Weitz J I. 1998. Characterization of the interactions of plasminogen and tissue and vampire bat plasminogen activators with fibrinogen, fibrin, and the complex of D-dimer noncovalently linked to fragment E. J Biol Chem 273:18292-18299.
Traynelis, S F, Lipton, S A. 2001. Is tissue plasminogen activator a threat to neurons? Nature Medicine, 7:17-18.
Toschi L, Bringmann P, Petri T, Donner P, Schleuning W D. 1998. Fibrin selectivity of the isolated protease domains of tissue-type and vampire bat salivary gland plasminogen activators. Eur J Biochem 252:108-112.
Tsirka S E, Gualandris A, Amaral D G, Strickland S. 1995. Excitotoxin-induced neuronal degeneration and seizure are mediated by tissue plasminogen activator. Nature. 377:340-344.

Tsirka, S., Rogove, A. D., and Strickland, S. 1996. Neuronal cell death and tPA. Nature 384: 123-124
Tsirka S E, Rogove A D, Bugge T H, Degen J L, Strickland S. 1997. An extracellular proteolytic cascade promotes neuronal degeneration in the mouse hippocampus. J Neurosci 17:543-552
Tsirka S E, Bugge T H, Degen J L, Strickland S. 1997. Neuronal death in the central nervous system demonstrates a non-fibrin substrate for plasmin. Proc Natl Acad Sci USA 94:9779-9781.
Wang Y F, Tsirka S E, Strickland S, Stieg P E, Soriano S G, Lipton S A. 1998. Tissue plasminogen activator (tPA) increases neuronal damage after focal cerebral ischemia in wild-type and tPA-deficient mice. Nat. Med. 4:228-231.
Walker J B, Nesheim M E. 2001. A kinetic analysis of the tissue plasminogen activator and DSPAα1 cofactor activities of untreated and TAFIa-treated soluble fibrin degradation products of varying size. J Biol Chem 276:3138-3148.
(8) Warach S. Thrombolysis in stroke beyond three hours: Targeting patients with diffusion and perfusion MRI. Ann Neurol. 2002; 51:11-13.
Witt W, Maass B, Baidus B, Hildebrand M, Donner P, Schleuning W D. 1994. Coronary thrombolysis with Desmodus salivary plasminogen activator in dogs. Fast and persistent recanalization by intravenous bolus administration. Circulation. 90:421-426.

We claim:

1. A method of treating stroke in a mammal, comprising administering to the mammal a composition comprising DSPAα1 in an amount from 60 to 230 μg/kg body weight of the mammal.

2. The method of claim 1, wherein the amount of DSPAα1 is from 62.5 to 130 μg/kg body weight of the mammal.

3. The method of claim 2, wherein the amount of DSPAα1 is from 90 to 130 μg/kg body weight of the mammal.

4. The method of claim 3, wherein the amount of DSPAα1 is 125 μg/kg body weight of the mammal.

5. The method of claim 1, wherein the drug is administered to the mammal intravenously.

6. The method of claim 1, wherein the drug is administered to the mammal as a bolus injection.

7. The method according to claim 1, wherein DSPAα1 is administered from at least 3 hours after onset of a stroke.

8. The method according to claim 1, wherein DSPAα1 is administered from at least 6 hours after onset of a stroke.

9. The method according to claim 1, wherein DSPAα1 is administered from at least 9 hours after onset of a stroke.

10. The method of claim 4, wherein the drug is administered to the mammal intravenously.

11. The method of claim 4, wherein the drug is administered to the mammal as a bolus injection.

12. The method according to claim 4, wherein DSPAα1 is administered from at least 3 hours after onset of a stroke.

13. The method according to claim 4, wherein DSPAα1 is administered from at least 6 hours after onset of a stroke.

14. The method according to claim 4, wherein DSPAα1 is administered from at least 9 hours after onset of a stroke.

15. A method for producing a non-neurotoxic plasminogen activating factor comprising at least one of the following steps for the modification of the plasminogen activating factor:
introducing at least a part of a cymogenic triade into the plasminogen activating factor;
substituting Asp194 or a homologous aspartate in the plasminogen activating factor;
substituting the hydrophobic amino acid residues in the autolysis loop or in homologous peptide sections of the plasminogen activating factor; or
introducing a mutation in the cymogene for preventing the catalysis of the cymogene by plasmin.

16. A modified plasminogen activating factor produced according to the modification recited in the method of claim 15.


SEQUENCE LISTING (3 sequences)

SEQ ID NO: 1 - protein, 476 aa, Artificial Sequence
  Description of Artificial Sequence Synthetic modified tissue plasminogen activator
MVNTMKTKLLCVLLLCGAVFSLPRQETYRQLARGSRAYGVACKDEITQMTYRRQESWLRPEVRSKRVEHCQCDRGQARCHTVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSSTCGLRQYSQPQFHSTGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 2 - protein, 477 aa, Artificial Sequence
  Description of Artificial Sequence Synthetic modified urokinase
MVNTMKTKLLCVLLLCGAVFSLPRQETYRQLARGSRAYGVACKDEITQMTYRRQESWLRPEVRSKRVEHCQCDRGSNELHQVPSNSCDEPRCLNGGTCVSNKYFSIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADFQCGQKTLREPRFHSTGGEFTTIENQPWFAAIYRRHRGGSGVTYVCGGSLMSPCWVISATHCFIDYPKKEDYIVYLGRSRLNSNTQGEMKFEVENLILHKDYSADTHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKEIYPNVTDSCQGDSGGPLVCSLQGRMTLTGIVSWGRGCALKDKPGVYTRVSHFLPWIRSHTKL

SEQ ID NO: 3 - protein, 4 aa, Artificial Sequence
  Description of Artificial Sequence Synthetic peptide
GPRP